(19) United States
(12) Patent Application Publication
(10) Pub. No.: US 2026/0109764 A1
(43) Pub. Date: Apr. 23, 2026

(54) MONOVALENT CD47 BINDING PROTEINS

(71) Applicant: Caedo Oncology AS, Oslo (NO)

(72) Inventors: Kjetil Hestdal, Oslo (NO); Rolf Dagfinn Pettersen, Oslo (NO); Nina Richartz, Oslo (NO); Sittana Matar, Oslo (NO); Seham Skah, Oslo (NO)

(73) Assignee: Caedo Oncology AS, Oslo (NO)

(21) Appl. No.: 19/118,115

(22) PCT Filed: Oct. 9, 2023
(86) PCT No.: PCT/EP2023/077918

§ 371 Date: Apr. 3, 2025

(30) Foreign Application Priority Data
Oct. 7, 2022 (EP) 22200439.2
Mar. 20, 2023 (GB) 2304051.2

(51) Int. Cl. C07K 16/28; A61K 39/00; A61K 47/68; A61P 35/00

(52) U.S. Cl. CPC C07K 16/2803; A61K 47/6849; A61P 35/00; A61K 2039/505; C07K 2317/35; C07K 2317/52; C07K 2317/565; C07K 2317/569; C07K 2317/73; C07K 2317/92

(57) ABSTRACT

The present invention relates to binding protein or antibody comprising one antigen binding domain that binds to CD47, said antigen binding domain comprising a heavy chain variable region that comprises three complementarity determining regions (CDRs), and a light chain variable region that comprises three CDRs, wherein said antigen binding domain binds to Q19, N45, T120, R121, E122 and G123 within CD47 as defined by SEQ ID NO:19, and wherein said binding protein or antibody binds monovalently to CD47. Binding protein and antibody-based compositions, therapeutic methods and kits are also provided.

This invention relates generally to the field of binding proteins that bind monovalently to CD47, in particular antibodies, and in particular binding proteins and antibodies, for example antibody fragments, that bind monovalently to human CD47. Binding protein and antibody-based compositions, methods and kits are also provided. Such anti-CD47 antibodies have therapeutic uses, such as the treatment or diagnosis of cancer. Binding protein and antibody-based compositions, methods and kits are also provided.

The treatment of cancer is still one of the biggest unmet medical needs to date. While there have been advances in cancer therapy during the last decades, cancer remains one of the leading causes of death. As the populations in the industrialized countries are benefitting from longer average life expectancies, the urgency for improved or new cancer therapies is increasing.

A relatively new approach is to target the CD47 axis. CD47 is a ubiquitously expressed cell surface glycoprotein that functions as a signalling receptor for thrombospondin-1 and as the counter-receptor for signal regulatory protein-α (SIRP-α). Engaging SIRP-α on macrophages inhibits phagocytosis and CD47 thereby serves as a physiological marker of self. However, elevated CD47 expression on some cancer cells also protects cancer cells from innate immune surveillance and can act to prevent phagocytosis of cancer cells by SIRPα expressing macrophages and other cells of the innate immune system (the so called “don't eat me” signal).

These findings have led to the development of antibodies and other types of biologics to inhibit CD47/SIRPα interactions in tumor cells. There are several candidate molecules in preclinical and clinical development, for example from Gilead, FortySeven, ALX Oncology and Arch Oncology. However, there is still a need for alternative and preferably improved therapeutics which target CD47.

The present invention provides one such alternative and improved therapeutic option in the form of binding proteins and antibodies/antibody fragments (e.g. antibody based binding proteins) directed to CD47 and that bind monovalently to CD47.

As will be described in more detail elsewhere herein, antibodies of the invention, including humanized antibodies of the invention, have been shown to be capable of binding to CD47 with high affinity and surprisingly also show excellent ability to induce direct killing of tumour cells. Advantageously the direct cell killing effects are observed rapidly and also with very low concentrations. Antibodies/antibody fragments of the invention also surprisingly do not induce haemagglutination even at high concentrations.

To the inventors' knowledge no other anti-CD47 antibodies have been disclosed to have this advantageous combination of properties and antibodies (or binding proteins) with one or more, preferably all, of these properties are preferred.

Such antibodies of the invention (or for example other binding proteins of the invention comprising a CD47 antigen binding domain as described herein) can conveniently and advantageously be used for the treatment of diseases associated with CD47 expression, in particular for the treatment of cancer.

In one embodiment, the present invention provides a binding protein, for example an antibody, comprising one antigen binding domain that binds to CD47, said antigen binding domain comprising a heavy chain variable region that comprises three complementarity determining regions (CDRs), and a light chain variable region that comprises three CDRs, wherein said heavy chain variable region comprises:
(i) a variable heavy (VH) CDR1 that comprises the amino acid sequence of NFGMH (SEQ ID NO:5) or a sequence substantially homologous thereto,
(ii) a VH CDR2 that comprises the amino acid sequence of WINTYTGEPTYTDDFKG (SEQ ID NO:6) or a sequence substantially homologous thereto, and
(iii) a VH CDR3 that comprises the amino acid sequence of GDYRYGDS (SEQ ID NO:7) or a sequence substantially homologous thereto; and/or
wherein said light chain variable region comprises:
(iv) a variable light (VL) CDR1 that comprises the amino acid sequence of RSSQSLVHSNGKTYLH (SEQ ID NO:8) or a sequence substantially homologous thereto,
(v) a VL CDR2 that comprises the amino acid sequence of RVSNRFS (SEQ ID NO:9) or a sequence substantially homologous thereto, and
(vi) a VL CDR3 that comprises the amino acid sequence of SQSTHVPFT (SEQ ID NO:10) or a sequence substantially homologous thereto;
wherein said substantially homologous sequence is a sequence containing 1, 2 or 3 amino acid substitutions compared to the given CDR sequence; and
wherein said binding protein binds monovalently to CD47.

In another embodiment, the present invention provides a binding protein, for example an antibody, comprising one antigen binding domain that binds to CD47, said antigen binding domain comprising a heavy chain variable region that comprises three complementarity determining regions (CDRs), and a light chain variable region that comprises three CDRs, wherein said heavy chain variable region comprises:
(i) a variable heavy (VH) CDR1 that comprises the amino acid sequence of NFGMH (SEQ ID NO:5) or a sequence substantially homologous thereto, wherein said substantially homologous sequence is a sequence containing 1 or 2 amino acid substitutions compared to the given CDR sequence,
(ii) a VH CDR2 that comprises the amino acid sequence of WINTYTGEPTYTDDFKG (SEQ ID NO:6) or a sequence substantially homologous thereto, wherein said substantially homologous sequence is a sequence containing 1, 2, 3, 4, 5 or 6, e.g. 1, 2, 3 or 4, amino acid substitutions compared to the given CDR sequence, and
(iii) a VH CDR3 that comprises the amino acid sequence of GDYRYGDS (SEQ ID NO:7) or a sequence substantially homologous thereto, wherein said substantially homologous sequence is a sequence containing 1, 2 or 3 amino acid substitutions compared to the given CDR sequence; and/or wherein said light chain variable region comprises:
(iv) a variable light (VL) CDR1 that comprises the amino acid sequence of RSSQSLVHSNGKTYLH (SEQ ID NO:8) or a sequence substantially homologous thereto, wherein said substantially homologous sequence is a sequence containing 1, 2, 3, 4, 5 or 6, e.g. 1, 2, 3 or 4, amino acid substitutions compared to the given CDR sequence,
(v) a VL CDR2 that comprises the amino acid sequence of RVSNRFS (SEQ ID NO:9) or a sequence substantially homologous thereto, wherein said substantially homologous sequence is a sequence containing 1, 2 or 3 amino acid substitutions compared to the given CDR sequence, and
(vi) a VL CDR3 that comprises the amino acid sequence of SQSTHVPFT (SEQ ID NO:10) or a sequence substantially homologous thereto, wherein said substantially homologous sequence is a sequence containing 1, 2 or 3 amino acid substitutions compared to the given CDR sequence; and wherein said binding protein binds monovalently to CD47.

In another embodiment, the present invention provides a binding protein, for example an antibody, comprising one antigen binding domain that binds to CD47, said antigen binding domain comprising a heavy chain variable region that comprises three complementarity determining regions (CDRs), and a light chain variable region that comprises three CDRs, wherein said heavy chain variable region comprises:
(i) a variable heavy (VH) CDR1 that comprises the amino acid sequence of NFGMH (SEQ ID NO:5),
(ii) a VH CDR2 that comprises the amino acid sequence of WINTYTGEPTYTDDFKG (SEQ ID NO:6), and
(iii) a VH CDR3 that comprises the amino acid sequence of GDYRYGDS (SEQ ID NO:7); and/or wherein said light chain variable region comprises:
(iv) a variable light (VL) CDR1 that comprises the amino acid sequence of RSSQSLVHSNGKTYLH (SEQ ID NO:8),
(v) a VL CDR2 that comprises the amino acid sequence of RVSNRFS (SEQ ID NO:9), and
(vi) a VL CDR3 that comprises the amino acid sequence of SQSTHVPFT (SEQ ID NO:10); and
wherein said binding protein binds monovalently to CD47.

In a preferred embodiment, the present invention provides a binding protein, for example an antibody, comprising one antigen binding domain that binds to CD47, said antigen binding domain comprising a heavy chain variable region that comprises three complementarity determining regions (CDRs), and a light chain variable region that comprises three CDRs, wherein said heavy chain variable region comprises:
(i) a variable heavy (VH) CDR1 that comprises the amino acid sequence of NFGMH (SEQ ID NO:5),
(ii) a VH CDR2 that comprises the amino acid sequence of WINTYTGEPTYTDDFKG (SEQ ID NO:6), and
(iii) a VH CDR3 that comprises the amino acid sequence of GDYRYGDS (SEQ ID NO:7); and wherein said light chain variable region comprises:
(iv) a variable light (VL) CDR1 that comprises the amino acid sequence of RSSQSLVHSNGKTYLH (SEQ ID NO:8),
(v) a VL CDR2 that comprises the amino acid sequence of RVSNRFS (SEQ ID NO:9), and
(vi) a VL CDR3 that comprises the amino acid sequence of SQSTHVPFT (SEQ ID NO:10); and
wherein said binding protein binds monovalently to CD47.

Put another way, the present invention provides a binding protein, for example an antibody, comprising one antigen binding domain that binds to CD47, said antigen binding domain comprising a heavy chain variable region that comprises three complementarity determining regions (CDRs), and a light chain variable region that comprises three CDRs, wherein said heavy chain variable region comprises:
(i) a variable heavy (VH) CDR1 that comprises the amino acid sequence of NFGMH (SEQ ID NO:5) or a sequence substantially homologous thereto,
(ii) a VH CDR2 that comprises the amino acid sequence of WINTYTGEPTYTDDFKG (SEQ ID NO:6) or a sequence substantially homologous thereto, and
(iii) a VH CDR3 that comprises the amino acid sequence of GDYRYGDS (SEQ ID NO:7) or a sequence substantially homologous thereto; and/or wherein said light chain variable region comprises:
(iv) a variable light (VL) CDR1 that comprises the amino acid sequence of RSSQSLVHSNGKTYLH (SEQ ID NO:8) or a sequence substantially homologous thereto,
(v) a VL CDR2 that comprises the amino acid sequence of RVSNRFS (SEQ ID NO:9) or a sequence substantially homologous thereto, and
(vi) a VL CDR3 that comprises the amino acid sequence of SQSTHVPFT (SEQ ID NO:10) or a sequence substantially homologous thereto;
wherein said substantially homologous sequence is a sequence containing 1, 2 or 3 amino acid substitutions compared to the given CDR sequence.

Other features of the antibodies and binding proteins as described herein apply equally, mutatis mutandis, to this embodiment.

Such binding proteins of the invention, preferably antibodies, are thus monovalent for CD47 (or can bind monovalently to CD47), e.g. can comprise 1 antigen binding domain that binds to CD47 (e.g. only one antigen binding domain that binds to CD47). Further such exemplary binding proteins of the invention, preferably antibodies, may however have additional antigen binding domains that bind to target antigens other than CD47. Thus, such binding proteins (or antibodies) can be bi-specific, tri-specific or multi-specific, i.e. bind to more than one type of target antigen, wherein one of the target antigens is CD47. Thus, such binding proteins (or antibodies) still require one antigen binding domain that binds monovalently to CD47. Exemplary such binding proteins (or antibodies) have one, or only only, antigen binding domain, or 6, or only 6, CDRs (e.g. one set of 6 CDRs) specific for CD47.

In a further embodiment, the present invention provides a monovalent binding protein, for example an antibody, comprising one antigen binding domain that binds to CD47, said antigen binding domains comprising a heavy chain variable region that comprises three complementarity determining regions (CDRs), and a light chain variable region that comprises three CDRs, wherein said heavy chain variable region comprises:
(i) a variable heavy (VH) CDR1 that comprises the amino acid sequence of NFGMH (SEQ ID NO:5) or a sequence substantially homologous thereto,
(ii) a VH CDR2 that comprises the amino acid sequence of WINTYTGEPTYTDDFKG (SEQ ID NO:6) or a sequence substantially homologous thereto, and
(iii) a VH CDR3 that comprises the amino acid sequence of GDYRYGDS (SEQ ID NO:7) or a sequence substantially homologous thereto; and/or wherein said light chain variable region comprises:
(iv) a variable light (VL) CDR1 that comprises the amino acid sequence of RSSQSLVHSNGKTYLH (SEQ ID NO:8) or a sequence substantially homologous thereto,
(v) a VL CDR2 that comprises the amino acid sequence of RVSNRFS (SEQ ID NO:9) or a sequence substantially homologous thereto, and
(vi) a VL CDR3 that comprises the amino acid sequence of SQSTHVPFT (SEQ ID NO:10) or a sequence substantially homologous thereto;
wherein said substantially homologous sequence is a sequence containing 1, 2 or 3 amino acid substitutions compared to the given CDR sequence.

The term “monovalent” as used herein refers to a binding protein (or antibody) with one antigen binding domain. The term “monovalent for CD47”, or equivalent terms, refers to a binding protein (or antibody) with one antigen binding domain which can bind to one molecule of the same target antigen, here CD47. Such binding proteins or antibodies may be “monovalent for CD47”, but may also include additional antigen binding domains that bind to target antigens other than CD47. Thus, such binding proteins (or antibodies) can be bi-specific, tri-specific or multi-specific, i.e. bind to more than one type of target antigen, wherein one of the target antigens is CD47.

Thus, the binding proteins (or antibodies) of the invention as described above and elsewhere herein are monovalent for CD47. Put another way, they can bind monovalently to CD47.

Certain embodiments of the invention provide an antibody (or binding protein) that binds to CD47, comprising a VH domain that has the amino acid sequence of SEQ ID NO: 3 or a sequence substantially homologous thereto, and/or a VL domain that has the amino acid sequence of SEQ ID NO: 4 or a sequence substantially homologous thereto.

Certain embodiments of the invention provide an antibody (or binding protein) that binds to CD47, comprising a VH domain that has the amino acid sequence of SEQ ID NO: 3 or a sequence substantially homologous thereto, and a VL domain that has the amino acid sequence of SEQ ID NO: 4 or a sequence substantially homologous thereto.

Certain embodiments of the invention provide an antibody (or binding protein) that binds to CD47, comprising a VH domain that has the amino acid sequence of SEQ ID NO: 3, and/or a VL domain that has the amino acid sequence of SEQ ID NO: 4.

Certain embodiments of the invention provide an antibody (or binding protein) that binds to CD47, comprising a VH domain that has the amino acid sequence of SEQ ID NO: 3, and a VL domain that has the amino acid sequence of SEQ ID NO: 4.

In another embodiment, the present invention provides an antibody (or binding protein) that binds to CD47, wherein the heavy chain variable region comprises the amino acid sequence of SEQ ID NO:3, or a sequence having at least 80% sequence identity thereto (e.g. at least 85%, 90%, 95% or 98%) and/or wherein the light chain variable region comprises the amino acid sequence of SEQ ID NO:4, or a sequence having at least 80% sequence identity thereto (e.g. at least 85%, 90%, 95% or 98%).

In another embodiment, the present invention provides an antibody (or binding protein) that binds to CD47, wherein the heavy chain variable region comprises the amino acid sequence of SEQ ID NO:3, or a sequence having at least 80% sequence identity thereto (e.g. at least 85%, 90%, 95% or 98%) and wherein the light chain variable region comprises the amino acid sequence of SEQ ID NO:4, or a sequence having at least 80% sequence identity thereto (e.g. at least 85%, 90%, 95% or 98%).

In another embodiment, the present invention provides an antibody (or binding protein) that binds to CD47, wherein the heavy chain variable region comprises the amino acid sequence of SEQ ID NO:3, or a sequence having at least 80% sequence identity thereto (e.g. at least 85%, 90%, 95% or 98%), further wherein said heavy chain variable region comprises three CDRs comprising the amino acid sequences of SEQ ID NO:5, 6 and 7, or sequences substantially homologous thereto, as defined elsewhere herein; and/or wherein the light chain variable region comprises the amino acid sequence of SEQ ID NO:4, or a sequence having at least 80% sequence identity thereto (e.g. at least 85%, 90%, 95% or 98%), further wherein said light chain variable region comprises three CDRs comprising the amino acid sequences of SEQ ID NO:8, 9 and 10, or sequences substantially homologous thereto, as defined elsewhere herein.

In another embodiment, the present invention provides an antibody (or binding protein) that binds to CD47, wherein the heavy chain variable region comprises the amino acid sequence of SEQ ID NO:3, or a sequence having at least 80% sequence identity thereto (e.g. at least 85%, 90%, 95% or 98%), further wherein said heavy chain variable region comprises three CDRs comprising the amino acid sequences of SEQ ID NO:5, 6 and 7, or sequences substantially homologous thereto, as defined elsewhere herein; and wherein the light chain variable region comprises the amino acid sequence of SEQ ID NO:4, or a sequence having at least 80% sequence identity thereto (e.g. at least 85%, 90%, 95% or 98%), further wherein said light chain variable region comprises three CDRs comprising the amino acid sequences of SEQ ID NO:8, 9 and 10, or sequences substantially homologous thereto, as defined elsewhere herein.

In another embodiment, the present invention provides an antibody (or binding protein) that binds to CD47, wherein the heavy chain variable region comprises the amino acid sequence of SEQ ID NO:3, or a sequence having at least 80% sequence identity thereto (e.g. at least 85%, 90%, 95% or 98%), further wherein said heavy chain variable region comprises three CDRs comprising the amino acid sequences of SEQ ID NO:5, 6 and 7; and/or wherein the light chain variable region comprises the amino acid sequence of SEQ ID NO:4, or a sequence having at least 80% sequence identity thereto (e.g. at least 85%, 90%, 95% or 98%), further wherein said light chain variable region comprises three CDRs comprising the amino acid sequences of SEQ ID NO:8, 9 and 10.

The above (and other) embodiments described for SEQ ID NO:3 and/or SEQ ID NO:4 apply equally to alternative heavy chain variable regions of the invention, e.g. SEQ ID NOs: 22, 23, 24, 25, or 26, and/or alternative light chain variable regions of the invention, e.g. SEQ ID NOs: 27, 28, or 29. Preferred pairings of such heavy and light chain variable regions are provided in Table B and elsewhere herein.

In another embodiment, the present invention provides an antibody (or binding protein) that binds to CD47, wherein the heavy chain variable region comprises the amino acid sequence of SEQ ID NO:3, or a sequence having at least 80% sequence identity thereto (e.g. at least 85%, 90%, 95% or 98%), further wherein said heavy chain variable region comprises three CDRs comprising the amino acid sequences of SEQ ID NO:5, 6 and 7; and wherein the light chain variable region comprises the amino acid sequence of SEQ ID NO:4, or a sequence having at least 80% sequence identity thereto (e.g. at least 85%, 90%, 95% or 98%), further wherein said light chain variable region comprises three CDRs comprising the amino acid sequences of SEQ ID NO:8, 9 and 10.

In alternative embodiments of the invention, the sequence identities can be sequences having at least 60%, 65%, 70% or 75%.

Anti-CD47 antibodies (and binding proteins) which bind monovalently to CD47 based on the CO-1 scFv antibody sequences set forth in Table A are preferred. The CDR domains, FR domains, VH and VL domains, and an exemplary scFv sequence (variable heavy and light chains and linker) are shown in Table A herein. Antibodies (or binding proteins) which bind monovalently to CD47 comprising these sets of CDR domains or VH and VL domains, or monovalent formats, e.g. scFv, comprising such domains (or sequences substantially homologous thereto), including the full scFv sequence provided in Table A are preferred embodiments of the invention. Humanized forms of the antibodies are also preferred, for example antibodies comprising a heavy chain variable domain that is a humanized version of SEQ ID NO:3 and/or a light chain variable domain that is a humanized version of SEQ ID NO:4. Such humanized versions include antibodies (or binding proteins) comprising the heavy and/or light chain variable domains as shown in Table B.

CDR sequences of certain antibodies of the invention are set forth herein in Table A. In some other embodiments, CDR sequences of antibodies of the invention may be CDR sequences in the VH domains and VL domains of antibodies of the invention as identified using any suitable method (or tool), for example as identified according to the well-known methods of Kabat (e.g. Kabat, et al., “Sequences of Proteins of Immunological Interest”, 5th Ed. Public Health Service, National Institutes of Health, Bethesda, MD, 647-669, 1991), e.g. as shown in Table A, or Chothia (e.g. Chothia C, et al. (1989) Nature, 342:877-883, or Al-Lazikani et al., (1997) JMB 273,927-948), or as identified using the IMGT numbering scheme (e.g. Lefranc, M.-P., The Immunologist, 7, 132-136 (1999); www.imgt.org)), or by AbM numbering (e.g. Abhinandan and Martin, 2008, Mol. Immunol. 45:3832-3839).

Certain examples of substantially homologous sequences are sequences that have at least 55%, 60% or 65% identity to the amino acid sequences disclosed. In certain embodiments, the antibodies (or binding proteins) of the invention comprise one (or only one) heavy chain variable region that includes an amino acid sequence region of at least 55%, 60%, 65%, 70% or 75%, more preferably at least 80%, more preferably at least 85%, more preferably at least 90% or 95% and most preferably at least 96%, 97%, 98% or 99% amino acid sequence identity to the amino acid sequence of SEQ ID NO: 3; and/or one (or only one) light chain variable region that includes an amino acid sequence region of at least 55%, 60%, 65%, 70% or 75%, more preferably at least 80%, more preferably at least 85%, more preferably at least 90% or 95% and most preferably at least 96%, 97%, 98% or 99% amino acid sequence identity to the amino acid sequence of SEQ ID NO: 4.

Other preferred examples of substantially homologous sequences are sequences containing conservative amino acid substitutions of the amino acid sequences disclosed.

Other preferred examples of substantially homologous sequences are sequences containing 1, 2, 3, 4, 5 or 6; 1, 2, 3, 4 or 5; 1, 2, 3 or 4, preferably 1, 2 or 3, preferably 1 or 2 (more preferably 1), altered amino acids in one or more of the CDR regions or one or more of the FR regions disclosed. Such alterations might be conserved or non-conserved amino acid substitutions, or a mixture thereof.

Other preferred examples of “substantially homologous” sequences are sequences having at least 50%, at least 55%, at least 60%, at least 65%, at least 70%, at least 75%, at least 80%, at least 85%, at least 90%, or at least 95%, amino acid sequence identity to the amino acid sequence of one or more of the CDR regions or one or more of the FR regions disclosed in Table A or B. Thus, in some embodiments, a “substantially homologous” CDR sequence may be a sequence having at least 50%, at least 55%, at least 60%, at least 65%, at least 70%, at least 75%, at least 80%, at least 85%, at least 90% or at least 95% sequence identity to a given CDR sequence described herein.

In some embodiments, in antibodies having a “substantially homologous” sequence as compared to a given sequence, or having a certain degree of sequence identity as compared to a given sequence, the altered amino acid residues(s) are not in a CDR region. For example, in some embodiments, in antibodies having a VH domain that has a certain degree of sequence identity to a given VH domain sequence of a particular antibody of the invention (e.g. CO-1 scFv, the humanized CO1-scFv antibodies of the invention, or the monovalent scFv-Fc fusion proteins), the altered (or variant) residue(s) are not in a CDR region. Thus, in some embodiments, in antibodies having a “substantially homologous” sequence as compared to a given sequence, or having a certain degree of sequence identity as compared to a given sequence, the altered amino acid residues(s) are in one or more framework regions.

As is evident from elsewhere herein, in other embodiments, in antibodies having a “substantially homologous” sequence as compared to a given sequence, or having a certain degree of sequence identity as compared to a given sequence, the altered amino acid residues(s) may be in a CDR region.

In some embodiments, in an antibody having a “substantially homologous” sequence as compared to a given sequence, or having a certain degree of sequence identity as compared to a given sequence, the three VH CDR amino acid sequences (i.e. all three VH CDR sequences taken together) and the three VL CDR amino acid sequences (i.e. all three VL CDR sequences taken together), to make up a set of six CDRs in total, are considered together to be the whole (or entire) CDR complement of the antibody, and the amino acid sequence of said whole CDR complement of said antibody is at least 70%, preferably at least 80%, or at least 85%, or at least 90%, or at least 95% identical to the corresponding whole (or entire) CDR complement of a given starting (or reference) antibody. The starting (or reference) antibody may have the CDR sequences of the CO-1 scFv antibody or the humanized CO1-scFv antibodies of the present invention as shown in Table A or B, or the CDR sequences of the monovalent scFv-Fc fusion protein (CO201-scFv-Fc-mono).

Altered residues might be conserved or non-conserved amino acid substitutions, or a mixture thereof.

In such embodiments, preferred alterations are conservative amino acid substitutions.

In all embodiments, binding proteins, e.g. antibodies, containing substantially homologous sequences retain the ability to bind monovalently to CD47. Preferably, binding proteins, e.g. antibodies, containing substantially homologous sequences retain one or more (preferably all) of the other properties described herein in relation to the antibodies of the invention, e.g. the CO-1 scFv antibody, the humanized CO1-scFv antibodies, or the monovalent scFv-Fc fusion proteins (e.g. CO201-scFv-Fc-mono) as described herein.

Further examples of substantially homologous amino acid sequences in accordance with the present invention are described elsewhere herein.

The CDRs of the antibodies (or binding proteins) of the invention are preferably separated by appropriate framework regions such as those found in naturally occurring antibodies and/or effective engineered antibodies. Thus, the VH. VL and individual CDR sequences of the invention are preferably provided within or incorporated into an appropriate framework or scaffold to enable antigen (here CD47) binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. In some embodiments, humanized antibodies are provided, in which case human framework regions (or sequences substantially homologous thereto) can be used. Alternatively, non-antibody scaffolds or frameworks, e.g. T cell receptor frameworks can be used.

Appropriate sequences that can be used for framework regions are well known and documented in the art and any of these may be used. Exemplary sequences for framework regions are one or more of the framework regions making up the VH, and/or VL domains of the antibodies of the invention, e.g. one or more of the framework regions of the CO-1 scFv antibody as disclosed in Table A, or one or more of the framework regions of the humanized CO-1 antibodies as disclosed in Table B, or framework regions substantially homologous thereto, and in particular framework regions that allow the maintenance of antigen specificity, for example framework regions that result in substantially the same or the same 3D structure of the antibody.

In certain embodiments, all four of the variable heavy chain (SEQ ID NOs: 11, 12, 13 and 14) and/or variable light chain (SEQ ID NOs: 15, 16, 17 and 18) framework regions (FR), as appropriate, or FR regions substantially homologous thereto, are found in the antibodies (or binding proteins) of the invention.

The exemplary CO-1 scFv antibody of the present invention comprises murine/mouse VH and VL domains (the antibody from which the scFv is derived, mCO-1, is a full length mouse (m) antibody; an IgG1 kappa antibody) and CO-1 scFv is a single chain format of this antibody in which a linker sequence has been placed between the VH and VL sequences. Such antibodies typically comprise murine/mouse VH and VL domains such as those with SEQ ID NO:s 3 and 4, or VH and VL domains which comprise heavy and light chain CDRs such as those with SEQ ID NO:s 5 to 10 (and related, e.g. substantially homologous sequences as described herein) separated by an appropriate linker sequence. scFv antibodies bind monovalently to CD47 and any other formats of antibody which allow such monovalent binding are also provided by the invention. Some examples are described elsewhere herein.

In other embodiments of the invention, humanized versions of the exemplary CO-1 antibodies of the present invention are preferred. Thus, where antibodies (or binding proteins) of the invention are referred to herein, preferred embodiments include humanized antibodies (or binding proteins).

Thus, in some embodiments, the antibodies (or binding proteins) of the invention can be, or can comprise, humanized antibodies, e.g. can be referred to as humanized antibodies or humanized binding proteins. “Humanized” antibodies, which are based on substantially non-human variable region domains, are antibodies in which certain amino acids have been changed to better correspond with the amino acids typically present in human antibodies. Methods for generating humanized antibodies are known in the art. For example, humanized antibodies can be generated by inserting the appropriate CDRs (e.g. murine/mouse CDRs such as those present in the antibodies of the present invention) into a human antibody “scaffold” such as a scaffold comprising human antibody framework regions or sequences substantially homologous thereto. Thus, in some embodiments, CDRs of the invention, e.g. a set of 6 CDRs of the antibodies of the invention as described herein, e.g. from the exemplary antibodies of the present invention as shown in Table A, i.e. CDRs with SEQ ID NO:s 5 to 10, or sequences substantially homologous thereto, are present within (or combined with or inserted or grafted into) a human or humanized antibody framework, e.g. using appropriate framework (FR) regions as found in human antibodies, or sequences substantially homologous thereto.

Exemplary humanized variable heavy (VH) domains for use in the humanized antibodies (or binding proteins) of the invention are provided in SEQ ID NOs: 22 to 26, or sequences substantially homologous thereto.

Exemplary humanized variable light (VL) domains for use in the humanized antibodies (or binding proteins) of the invention are provided in SEQ ID NOs: 27 to 29, or sequences substantially homologous thereto.

Preferred antibodies (or binding proteins), e.g. humanized antibodies (or binding proteins) of the invention thus comprise any one of the VH domains of SEQ ID NOs: 22, 23, 24, 25, or 26, or sequences substantially homologous thereto, and/or (preferably and) any one of the VL domains of SEQ ID NOs: 27, 28 or 29, or sequences substantially homologous thereto.

Thirteen humanized antibodies have been made in accordance with the present invention and these antibodies (or antibodies with sequences substantially homologous thereto), or binding proteins comprising such antibodies, are preferred antibodies (or binding proteins) of the present invention. These antibodies are referred to herein as:
CO201 (comprising a VH domain of SEQ ID NO: 22 and a VL domain of SEQ ID NO: 27);
CO202 (comprising a VH domain of SEQ ID NO: 23 and a VL domain of SEQ ID NO: 27);
CO203 (comprising a VH domain of SEQ ID NO: 24 and a VL domain of SEQ ID NO: 27);
CO204 (comprising a VH domain of SEQ ID NO: 25 and a VL domain of SEQ ID NO: 27);
CO205 (comprising a VH domain of SEQ ID NO: 22 and a VL domain of SEQ ID NO: 28);
CO206 (comprising a VH domain of SEQ ID NO: 23 and a VL domain of SEQ ID NO: 28);
CO207 (comprising a VH domain of SEQ ID NO: 24 and a VL domain of SEQ ID NO: 28);
CO208 (comprising a VH domain of SEQ ID NO: 25 and a VL domain of SEQ ID NO: 28);
CO209 (comprising a VH domain of SEQ ID NO: 22 and a VL domain of SEQ ID NO: 29);
CO210 (comprising a VH domain of SEQ ID NO: 23 and a VL domain of SEQ ID NO: 29);
CO211 (comprising a VH domain of SEQ ID NO: 24 and a VL domain of SEQ ID NO: 29);
CO212 (comprising a VH domain of SEQ ID NO: 25 and a VL domain of SEQ ID NO: 29); or
CO213 (comprising a VH domain of SEQ ID NO: 26 and a VL domain of SEQ ID NO: 27).

Other preferred humanized antibodies (or binding proteins) of the invention comprise a VH domain of SEQ ID NO: 26, or a sequence substantially homologous thereto, and a VL domain of SEQ ID NO: 28, or a sequence substantially homologous thereto; or a VH domain of SEQ ID NO: 26, or a sequence substantially homologous thereto, and a VL domain of SEQ ID NO: 29, or sequence substantially homologous thereto.

In some embodiments a humanized VH domain of SEQ ID NO:22 or a sequence substantially homologous thereto, is preferred. In some embodiments a humanized VL domain of SEQ ID NO:27, or a sequence substantially homologous thereto, is preferred.

In some embodiments a humanized VH domain of SEQ ID NO:22, or a sequence substantially homologous thereto, and a humanized VL domain of SEQ ID NO:27, or a sequence substantially homologous thereto, is preferred.

Sequences which are substantially homologous to any given sequence in such humanized antibodies (or binding proteins) are as defined elsewhere herein, and include sequences with varying numbers of amino acid substitutions, or with varying levels of percentage identity to a given starting sequence, e.g. sequences having at least 80% sequence identity thereto.

In some embodiments said variant residues may be present within both the CDR and FR regions of the antibodies (or binding proteins). In other embodiments said variant residues may be present within the CDR regions of the antibodies (or binding proteins). In other embodiments, said variant residues may be present within the FR regions of the antibodies (or binding proteins).

Preferred heavy chain FR regions found in the humanized antibodies (or binding proteins) of the present invention are one or more, or all four, of the FR regions FR1, FR2, FR3 and FR4 as present in SEQ ID NOs: 22, 23, 24, 25, or 26, or sequences substantially homologous thereto.

Preferred light chain FR regions found in the humanized antibodies (or binding proteins) of the present invention are one or more, or all four, of the FR regions FR1, FR2, FR3 and FR4 as present in SEQ ID NOs: 27, 28 or 29, or sequences substantially homologous thereto.

In certain embodiments, all four of the heavy chain and/or light chain framework regions (FR), as appropriate, from SEQ ID NOs: 22, 23, 24, 25, 26, 27, 28 or 29, or FR regions substantially homologous thereto, are found in the antibodies (or binding proteins) of the invention.

In embodiments where FR regions that are substantially homologous to one or more of the FR regions provided in SEQ ID NOs: 22, 23, 24, 25, 26, 27, 28 or 29 are used, then each FR region may contain up to 10 amino acid changes from the given sequence, for example 10, 9, 8, 7, 6, 5, 4, 3, 2 or 1 change(s). Although any amino acid changes may be used, in some embodiments said changes may be changes to revert said amino acid residues back to the residues found in the original murine antibody, here the CO-1 antibody. Such changes can also be referred to as back mutations. In some embodiments 1, 2 or 3 back mutations may be present in one or more of the FR regions provided in SEQ ID NOs: 22, 23, 24, 25, 26, 27, 28 or 29.

Such humanized VH and VL domains can be provided in any appropriate monovalent (for CD47) format in accordance with the present invention. Exemplary formats are discussed elsewhere herein but include scFv antibodies or Fab or Fab′ fragments and the VH and VL domains of these fragments can thus be humanized VH and VL domains, e.g. as described herein.

Other antibody (or binding protein) monovalent formats comprising antibody constant regions, and in particular human antibody constant regions, such as antibodies comprising an Fc region, e.g. Fc fusions, are also provided. Thus, particularly preferred antibodies (or binding proteins) of the invention have humanized (or other, e.g. non-humanized or murine) VH and VL domains of the invention as described herein, coupled to, fused or attached to an Fc region of an antibody. Such formats are still monovalent for CD47. Thus, a particularly preferred format is a monovalent scFv-Fc format/fusion in which one scFv antibody (e.g. a humanized scFv antibody) is fused or attached to an Fc region. Such formats are well known and described in the art and thus would preferably have one scFv fragment, e.g. one scFv fragment of the invention, that binds to CD47 fused or coupled or attached to an Fc region. In these embodiments preferably the Fc region is a human Fc region such as an IgG1 or IgG4 Fc region. Exemplary Fc regions comprise (or consist of, or consist essentially of) CH2 and CH3 domains, and optionally also a hinge domain.

Thus, in some embodiments the humanized (or other) VH domains and VL domains as described herein are provided in formats which are monovalent for CD47, scFv formats. In other words such antibodies (or binding proteins) of the invention contain a single scFv fragment that can bind to CD47. The scFv format is well known in the art and comprises (or consists of) a single polypeptide chain in which VH and VL domains of an antibody are connected by an appropriate peptide linker. Preferred and exemplary combinations of VH and VL domains for use in such monovalent scFv formats are described elsewhere herein (see for example Tables A and B) and for example the VH and/or VL domains, or the CDRs, from any CD47 antibody of the invention may be used. In some such embodiments, humanized VH and VL domains as described herein are preferred. In some such embodiments a humanized VH domain of SEQ ID NO:22 or a sequence substantially homologous thereto, is preferred. In some such embodiments a humanized VL domain of SEQ ID NO:27 or a sequence substantially homologous thereto, is preferred. In some embodiments a humanized VH domain of SEQ ID NO:22, or a sequence substantially homologous thereto, and a humanized VL domain of SEQ ID NO:27, or a sequence substantially homologous thereto, is preferred.

Appropriate linker sequences for use in such scFv fragments are well known and described in the art. Appropriate linker sequences are typically artificial and flexible linkers such as GS linkers. An exemplary GS linker sequence is provided as SEQ ID NO:20.

The monovalent scFv-Fc format (or monovalent scFv-Fc fusion protein) is engineered such that the scFv will be attached to one chain of the Fc region and the other chain of the Fc region will not have an antibody (or binding protein) attached. As the Fc region is a dimer then a monovalent format is provided through association of the two chains of the Fc region (i.e. the chain with an scFv and the “empty” chain). Exemplary Fc regions comprise CH2 and CH3 domains, and optionally also a hinge domain (or other appropriate linker, e.g. artificial linker or flexible linker), e.g. to connect the scFv to a chain of the Fc region. Preferably the Fc region is a human Fc region such as an IgG1 or IgG4 Fc region, although clearly appropriate hinge and CH2 and CH3 domains can be derived from other subtypes of antibodies, e.g. IgG2, etc, and such sequences are readily available in the art. An exemplary human IgG4 Fc region (CH2 and CH3 domains) is provided in SEQ ID NO:30. An exemplary hinge region is provided in SEQ ID NO:33. However, in order to encourage the correct association of the two chains making up the Fc, conveniently such fragments can conveniently be engineered using “knobs into hole” mutations. The sequence of one of the chains of this construct (the “knob” chain) is provided as SEQ ID NO:31 and comprises a hinge-CH2-CH3, together with an appropriate “knob” mutation. The sequence of the other chain of this construct (the “hole” chain) is provided as SEQ ID NO:32 and comprises a hinge-CH2-CH3 fragment, together with appropriate “hole” mutations. The scFv fragment is positioned at the N-terminal end of one of the chains. When these two heavy chain constructs are expressed the chains will dimerise and the complexes formed of knob-hole dimers can be selected which comprise a monovalent scFv-Fc construct, i.e. an Fc region with a single (monovalent) scFv (i.e. a monovalent scFv-Fc fusion). Exemplary and preferred fragments comprising the CO201 scFv fragment (CO201-scFv-Fc-mono) are shown in SEQ ID NOs: 34 (knob with CO201 scFv) and 35 (hole with hinge-CH2-CH3). It can be noted that the scFv fragment can equally be present on the “hole” chain rather than the “knob” chain.

In embodiments where an IgG4 hinge is used, such as that of SEQ ID NO:33, it may be desirable to make mutations, for example stabilizing mutations, e.g. to prevent Fab-arm exchange (Handlogten et al., 2020, MABS 12(1), e1779974). Three exemplary mutations have been identified in the form of Y219C, G220C and S228P (see SEQ ID NO:36), and one or more of these may be used in the IgG4 hinge region.

In a similar way to the monovalent scFv-Fc format, other exemplary formats do not contain other antibody constant regions beyond the CH2 and CH3 heavy chain regions/domains. Thus, in some embodiments a CH1 region and/or a CL (or CL1) region is not present, or is removed. Alternatively viewed, in some embodiments, the only antibody constant regions present in the antibodies (or binding proteins) of the invention are the CH2 and CH3 regions (or an Fc region), optionally with a hinge domain (or other appropriate linker, e.g. artificial linker or flexible linker).

Thus, preferred such constructs contain antibodies (or binding proteins) of the invention as defined herein, e.g. have one scFv fragment which comprises VH and/or VL domains of the invention or the corresponding three or six, e.g. the six, CDRs of the invention as described elsewhere herein.

A preferred antibody (or binding protein) for use in such formats is the humanized CO201 antibody as described herein. However any CD47 antibody (or binding protein), e.g. any CD47 antibody (or binding protein) of the invention, e.g. any other humanized antibody of the invention, or the VH and/or VL domains (or their corresponding CDRs), or the six CDRs as shown in Table A (with appropriate FR regions), can be incorporated into this format and antibodies (or binding proteins) in this format or comprising this format (a monovalent scFv-Fc format), are preferred.

A yet further aspect of the invention provides an antibody (or binding protein) comprising one antigen binding domain that binds to CD47, wherein said antigen binding domain is in an scFv format, and wherein said antigen binding domain is fused or connected or otherwise attached to an Fc region (e.g. via a hinge region or linker). Thus, in these embodiments a CH1 region and/or a CL (or CL1) region is not present, or is removed, and such regions are not involved in linking or connecting the scFv to the Fc region. Thus, in these embodiments, said one scFv antigen binding domain can be directly attached to the Fc region, optionally via a hinge region or linker. In other words such antibodies (or binding proteins) are, or consist of, or comprise monovalent scFv-Fc fragments comprising one scFv antigen binding domain that binds to CD47. As mentioned above, preferred scFv antigen binding domains that bind to CD47 comprise one or more antigen binding domains of the invention as defined elsewhere herein. As in other aspects of the invention, such antibodies (or binding proteins) comprising Fc fusions bind monovalently to CD47.

Such antibodies (or binding proteins) can also readily be bi-specific, tri-specific or multi-specific constructs as described elsewhere herein. For example, antigen binding domains (conveniently in the form of scFv fragments) with specificity for target antigens other than CD47 can be present in such constructs, e.g. attached to the CH3 part of the Fc region. Such antibodies (or binding proteins) of the invention, when provided in a monovalent scFv-Fc format, surprisingly and advantageously have been shown to not induce haemagglutination of red blood cells, even at high concentrations. Thus, this particular format is believed to be a particularly advantageous format for CD47 antibodies and preferred antibodies (or binding proteins) of the invention, including humanized antibodies of the invention, when provided in a monovalent scFv-Fc format, do not induce haemagglutination (or significant haemagglutination) of RBCs when used at a concentration of, or at least, or up to 1, 2, 5 or 10 μg/ml. Preferred such antibodies (or binding proteins) of the invention do not induce haemagglutination (or significant haemagglutination) of RBCs when used at a concentration of, or at least, or up to 15, 20, 25, 50, 75 or 100 μg/ml.

Other features and properties, e.g. preferred features and properties, of other aspects of the invention, e.g. binding affinities, can apply, mutatis mutandis, to this aspect of the invention.

Advantageously such formats also have the ability to induce phagocytosis. This provides an improvement over other monovalent formats.

Thus, in preferred embodiments, antibodies (or binding proteins) of the invention (including humanized antibodies or binding proteins of the invention), when provided in a monovalent scFv-Fc format, can result in phagocytosis of CD47 expressing tumour cells by macrophages.

Thus, in some embodiments the antibodies (or binding proteins) of the invention (including humanized antibodies or binding proteins of the invention), when provided in a monovalent scFv-Fc format, can inhibit (or block) the “don't eat me” signal from CD47 expressed on tumour cells which then can result in phagocytosis of the tumour cells by SIRPα expressing macrophages or other innate immune cells.

The ability of the antibodies (or binding proteins) of the invention to induce such phagocytosis of tumour cells (e.g. CD47 expressing tumour cells) is advantageous. The monovalent scFv-Fc format antibodies of the invention have been shown to be particularly effective in inducing this phagocytosis, for example have been shown to able to induce phagocytosis of a significant number/percentage of tumour cells when in the presence of macrophages. In addition, they have been shown to demonstrate this effect at a relatively low dose or concentration.

Phagocytosis can conveniently be measured using appropriate in vitro assays which would be well known to a person skilled in the art. Such assays generally involve bringing target cells (here CD47 expressing tumor cells) into contact with macrophages in the presence of test antibodies (or binding proteins) of the invention, and assessing the number (e.g. as a percentage) of tumor cells that are subject to phagocytosis. A preferred assay is described in the Examples in which macrophages (e.g. mouse macrophages such as RAW 264.7 cells) and tumor cells are each stained by a different dye and then brought into contact with each other in the presence of test concentrations of an antibody being tested. At the end of the assay the cells are analysed by flow cytometry. Double stained cells represent phagocytosed target cells and the number of these can conveniently be expressed as a percentage.

In some embodiments, monovalent scFv-Fc format antibodies of the invention can induce phagocytosis at concentrations of 10 μg/ml. However, preferred antibodies of the invention can induce phagocytosis at concentrations of less than 10 μg/ml, 5 μg/ml, 2 μg/ml or 1 μg/ml. Further preferred monovalent scFv-Fc format antibodies of the invention can induce phagocytosis at concentrations of, or less than, 0.1 or 1 μg/ml, e.g. from between 0.1 to 1 μg/ml.

The antibodies (or binding proteins) of the invention are preferably capable of inducing phagocytosis of a range of cancer cells, for example cells from haematological cancers and also from solid tumours. Purely by way of example, and not meant to provide an exhaustive list, the antibodies (or binding proteins) of the invention have been shown to be capable of inducing phagocytosis of haematological cancer cells such as Jurkat cells.

The % phagocytosis values will naturally vary depending on the cell type concerned. However, the exemplified monovalent scFv-Fc format antibodies of the invention show good % phagocytosis levels to cancer cells, for example levels of at least 10% phagocytosis can be seen for different types of cancer cells, with levels of at least 20%, 25%, 30% or 35% being observed for some types of cancer cells. Purely by way of example, monovalent scFv-Fc format antibodies (or binding proteins) of the invention can induce at least, or up to, 20%, 25%, 30%, or 35%, phagocytosis of Jurkat cells (a cancerous T cell line), for example when the cells were exposed for 2 hours in the presence of macrophages to a monovalent scFv-Fc format antibody (e.g. CO201-scFv-Fc mono) at a concentration of 10, 1 or 0.1 μg/ml.

As described above, the present invention provides binding proteins, for example antibodies, or binding proteins (including humanized antibodies or binding proteins of the invention), comprising the antigen binding domain of an antibody, which bind to (or specifically recognise or specifically bind to) CD47, for example, human CD47, in a monovalent manner, i.e. with one or a single antigen binding domain that can bind to CD47. Preferred binding proteins of the invention are antibodies. However, embodiments as described herein which relate to antibodies, apply equally, mutatis mutandis, to other types of binding proteins, or vice versa. Thus, other binding proteins can comprise the antibodies of the invention or can comprise the antigen binding domains of the antibodies of the invention, e.g. the three VL CDR regions and/or the three VH CDR regions of the antibodies of the invention, or a VL and/or VH domain (i.e. three CDR regions (CDR1, CDR2 and CDR3) and the four FR regions (FR1, FR2, FR3 and FR4)) of the VL and/or VH domains of the antibodies of the invention.

Preferred binding proteins are any polypeptide chains which can bind (e.g. specifically bind) to CD47, for example human CD47, in a monovalent manner, i.e. with one or a single antigen binding domain that can bind to CD47. Appropriate types of binding protein which could be used in the invention are known in the art. For example, in some embodiments immunoglobulin based polypeptides are used, which generally comprise CDR regions (and optionally FR regions or an immunoglobulin based scaffold), such that the CDR regions (and optionally FR regions) of the antibodies of the invention can be grafted onto an appropriate scaffold or framework, e.g. an immunoglobulin scaffold. Alternatively, the antigen binding fragments or the antibodies of the invention can be incorporated into any appropriate antigen binding fragment or antibody containing format, e.g. can be incorporated into a chimeric antigen receptor (CAR) format or a CAR-T cell format.

As described above, the present invention provides antibodies (or binding proteins), for example isolated antibodies (or binding proteins), which bind to (or specifically recognise or specifically bind to) CD47. CD47 is also sometimes referred to as Integrin Associated protein (IAP), MER6 or OA3. CD47 is expressed on all cell types but is strongly expressed or overexpressed on the surface of a variety of cancer cells, for example non-Hodgkin's lymphoma, Burkitt's lymphoma, acute myeloid leukemia (AML), hepatocellular carcinoma and bladder cancer. High expression is associated with poor prognosis in several cancer types, e.g. AML.

In accordance with the present invention, the CD47 may be from any species. In a preferred embodiment the CD47 is human CD47. Thus, in certain embodiments the antibodies (or binding proteins) of the invention can bind to human CD47. CD47 is a recognised target for cancer therapy.

The binding proteins or antibodies of the present invention thus bind or are capable of binding to CD47, e.g. human CD47.

The binding proteins and antibodies of the invention can bind to any appropriate forms of CD47. Preferred and convenient forms of CD47 to which the binding proteins and antibodies of the invention can bind include recombinant CD47, e.g. a recombinant human CD47, or a native or natural form of CD47, for example CD47 when present on the cell surface (cell-surface CD47), for example CD47 as expressed on tumor or cancer cells.

The sequences of CD47, e.g. human CD47, are well known and described in the art and can be obtained for example from various sequence databases, e.g. Uniprot entry Q08722 provides sequences of human CD47. Recombinant human CD47 is commercially available.

An appropriate and exemplary human CD47 sequence is provided below as SEQ ID NO:19. Thus, preferred binding proteins or antibodies of the invention bind to or are capable of binding (or specifically binding) to SEQ ID NO:19, or a sequence substantially homologous thereto (e.g. with at least 80% identity thereto), or a fragment, e.g. a biologically active fragment, thereof.

(SEQ ID NO: 19)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE

Methods of assessing binding to (or ability to bind to) appropriate forms of CD47 would be well-known to a person skilled in the art and any appropriate method can be used.

A convenient and appropriate method for assessing binding would include in vitro binding assays such as ELISA assays to assess binding of antibodies (or binding proteins) to immobilised antigen, such as immobilised forms of CD47 as described above, e.g. recombinant CD47, e.g. recombinant human CD47, e.g. comprising SEQ ID NO:19.

Thus, in certain embodiments, antibodies (or binding proteins) of the present invention can bind to CD47 in an ELISA assay. The skilled person will be familiar with ELISA assays and readily able to establish suitable conditions to assess the ability of an antibody to bind to CD47 in such an assay. For example, CD47 (e.g. recombinant human CD47) may be captured on ELISA plates, followed by washing and incubation with an anti-CD47 antibody (or binding protein) of the invention, and subsequent detection of bound anti-CD47 antibody (or binding protein). Typically, antibodies (or binding proteins) of the present invention can bind to human CD47 in an ELISA assay.

In certain embodiments, binding proteins or antibodies of the present invention bind to CD47 (e.g. recombinant CD47, e.g. recombinant human CD47, e.g. comprising SEQ ID NO:19) in (as determined in) a Surface Plasmon Resonance (SPR) assay (e.g. a BIACore assay, e.g. using a BIAcore S200 instrument). Suitable SPR assays are known in the art and for example may involve immobilising an antibody on a solid support and passing various concentrations of CD47 over the antibody. In certain preferred SPR assays, an appropriate form of CD47, e.g. recombinant CD47, e.g. recombinant human CD47, e.g. with SEQ ID NO:19, is captured (or immobilised) on a solid support (e.g. a sensor chip) and various concentrations (e.g. a dilution series, e.g. a doubling dilution series) of the binding protein or antibody to be tested is then injected. Antibody concentrations and RU Units are generally selected at a range and level, respectively, such that the chip is not saturated and which allow robust fitting, e.g. robust 1:1 fitting, by the SPR/Biacore software. Preferred concentrations and flow-rates for injection, together with appropriate RU Units are described in the Examples section.

Suitable association periods and dissociation periods to be used in an SPR assay are known to a skilled person, for example, a preferred association period in the SPR assay is 2 minutes and a preferred dissociation period in the SPR assay is 30 minutes (in a single cycle analysis). As described elsewhere herein, the antibodies of the invention can have a low off-rate, hence a relatively long dissociation period can be used. Thus, in a preferred embodiment, association may be measured over 2 minutes and/or dissociation may be measured over 30 minutes. In certain embodiments, all measurements may be performed at 25° C. in 20 mM PBS, pH7.4, 2.7 mM KCl, 137 mM NaCl, 0.05% P20. Kinetic parameters may be determined or calculated by any suitable model or software, for example by fitting the sensogram experimental data assuming a 1:1 interaction, in other words using a 1:1 binding model, for example using Single Cycle Kinetics software. Particularly preferred SPR assays are described in the Examples section herein. Preferably a single cycle analysis is used.

Thus, in some embodiments, antibodies (or binding proteins) of the invention are able to bind to CD47 (e.g. recombinant CD47, e.g. recombinant human CD47) in an SPR assay, or in an ELISA assay.

Such SPR assay methods can also conveniently be used to measure the binding kinetics of the antibody-antigen interaction, e.g. to determine association rate (ka), dissociation rate (kd) and affinity (KD).

In certain preferred embodiments, binding proteins or antibodies of the present invention, including humanized binding proteins or antibodies of the present invention, for example when in scFv format, or the monovalent scFv-Fc fusion proteins of the invention, or an alternative format which is monovalent for CD47 (e.g. a Fab format), have a high binding affinity for CD47 (e.g. human CD47, e.g. with SEQ ID NO:19), e.g. have a Ko (equilibrium dissociation constant) in the range of 750 pM or less, e.g. 750 pM or less, for example when determined in an SPR assay. Thus, preferably, binding proteins or antibodies of the invention, for example when in scFv format, or an alternative format which is monovalent for CD47 (e.g. a Fab format, or a monovalent scFv-Fc format), have a binding affinity for CD47 (e.g. human CD47, e.g. with SEQ ID NO:19) that is or corresponds to a K_{D} of less than 750 pM, preferably less than 700 pM, less than 650 pM, less than 600 pM or less than 550 pM, more preferably less than 500, 450, 400, 350, 300, 250, 200, 150, 125, 100, 75, 50 or 40 pM.

For example, the exemplified CO-1 scFv antibody of the invention shows a binding affinity of 180 pM, and the exemplary humanized antibodies of the invention show a binding affinity of 61 pM or less when in scFv format. In addition, the monovalent scFv-Fc fusion protein (CO201-scFv-Fc-mono) shows a binding affinity of 97 pM.

In some embodiments, antibodies of the present invention have an affinity for human CD47 that is higher than the affinity for human CD47 of certain comparator antibodies described in WO 2020/198370. Preferred affinities of antibodies of the invention are discussed elsewhere herein. In other embodiments, antibodies of the present invention have other advantageous properties, e.g. improved PCD as described elsewhere herein (e.g. higher levels of PCD, use of lower concentrations of antibodies, or faster induction), over certain comparator antibodies described in WO 2020/198370.

In some embodiments, the present invention provides a binding protein, for example an antibody, including humanized binding proteins or antibodies, comprising one antigen binding domain that binds to CD47, said antigen binding domain comprising a heavy chain variable region that comprises three complementarity determining regions (CDRs), and a light chain variable region that comprises three CDRs, wherein said binding protein or antibody binds monovalently to CD47, and wherein said binding protein or antibody has a binding affinity as defined elsewhere herein. Thus, preferably, such antibodies or binding proteins of the invention, for example when in scFv format, or an alternative format which is monovalent for CD47 (e.g. a Fab format or a monovalent scFv-Fc format), have a binding affinity for CD47 (e.g. human CD47, e.g. with SEQ ID NO:19) that is or corresponds to a K_{D} of less than 750 pM, preferably less than 700 pM, less than 650 pM, less than 600 pM or less than 550 pM, more preferably less than 500, 450, 400, 350, 300, 250, 200, 150, 125, 100, 75, 50 or 40 pM.

Any appropriate method of determining K_{D} may be used. However, preferably the K_{D} is determined in a Surface Plasmon Resonance assay (e.g. a BIAcore assay), preferably in which kinetic parameters are determined. Suitable and preferred types of SPR assay are described above. Thus, the K_{D} values as described above may be as determined in an SPR assay as described above or elsewhere herein, or K_{D} values observed when or if the antibodies of the invention are assessed in an SPR assay. Particularly preferred methods are described in the Example section herein.

Antibodies (or binding proteins) of the present invention (including humanized antibodies (or binding proteins) of the invention), can typically bind to cell-surface expressed CD47, such as cell-surface expressed human CD47 (CD47 expressed at the surface of cells, or present at or on the cell surface of CD47 expressing cells, e.g. human cells). Such cell-surface forms will thus in many cases represent a native or natural form of CD47 (or a native or natural configuration of CD47), for example the form found on cells which naturally express or overexpress CD47. CD47 is typically expressed at the surface of many tumour cells. In some embodiments, antibodies (or binding proteins) of the present invention bind to cell-surface expressed CD47 on human tumour cells. Binding to cell-surface CD47 can be assessed by any suitable means and preferred methods include by flow cytometry assays, e.g. as discussed elsewhere herein. In an exemplary flow cytometry method, CD47 expressing cells are incubated or contacted with the anti-CD47 antibody under investigation and the antibody bound to the CD47 on the cell is detected by fluorescence, for example the antibody is fluorescently labelled by an appropriate means, e.g. by direct or indirect labelling. Accordingly, if the anti-CD47 antibody under investigation binds to CD47 on the cell surface, the cell becomes fluorescently labelled and such cells, and thus antibodies (or binding proteins) which have the ability to bind to cell surface CD47, can be readily identified using a flow cytometer. A particularly preferred flow cytometry method is described in the Examples herein. Another method for testing for the ability of an antibody to bind to CD47 on the cell surface is immunohistochemistry.

EC₅₀ values can be used to quantify the binding of the antibodies (or binding proteins) of the invention to CD47 as expressed on tumour cells. Methods of calculating EC₅₀ values would be well known to a skilled person. However, conveniently the EC₅₀ values herein can be determined by, or when determined by, flow cytometry assays, for example by incubating or contacting an appropriate cell line with increasing concentrations of antibodies of the invention directly or indirectly conjugated to a fluorescent label (conveniently a FITC conjugate), followed by analysis by flow cytometry. An exemplary antibody (or binding protein) concentration range used herein is 0.1 ng/ml to 100 ug/ml. Appropriate curve fitting can then be carried out using appropriate software, for example using GraphPad Prism.

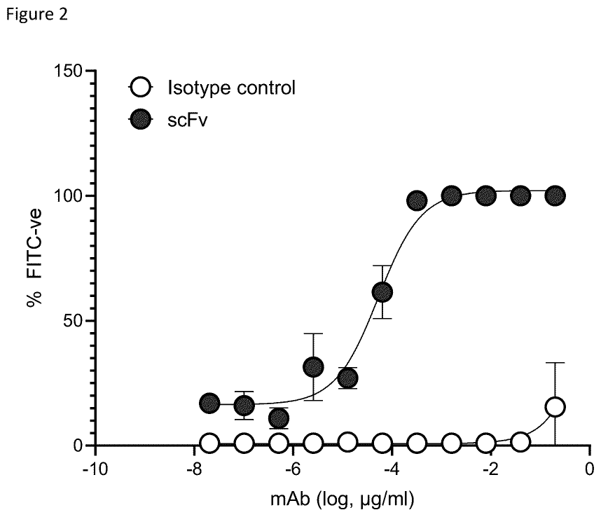

The antibodies (or binding proteins) of the invention (including humanized antibodies or binding proteins of the invention) are preferably capable of binding to a wide range of cancer cells, for example cells from haematological cancers and also from solid tumours. Purely by way of example, the antibodies (or binding proteins) of the invention have been shown to be capable of binding to Jurkat T cells, which is an example of haematological cancer cells (see FIG. 2). The EC₅₀ values will naturally vary depending on the cell type concerned. However, the exemplified antibodies of the invention show extremely good binding to Jurkat cells, as can be seen by the low (ng/ml) EC₅₀ values reported in the Examples. Purely by way of example, antibodies (or binding proteins) of the invention can bind to Jurkat cells (a cancerous T cell line) with an EC₅₀ value of 5 ng/ml or less, preferably 1 ng/ml or less, or 0.1 ng/ml or less, or 0.06 ng/ml or less.

Thus, in certain embodiments, antibodies (or binding proteins) of the present invention have an EC₅₀ (e.g. for binding to cancer cells, e.g. CD47 expressing cancer cells, for example Jurkat T cells) of 5, 4, 3, 2 or 1 ng/ml or less, preferably 0.9, 0.8, 0.7, 0.6, 0.5, 0.4, 0.3, 0.2 or 0.1 ng/ml or less, more preferably 0.09, 0.08, 0.07, 0.06, 0.05, 0.04, 0.03, 0.02 or 0.01 ng/ml or less. In some embodiments, the EC₅₀ is 0.01, 0.03, 0.04, 0.05 or 0.1 to 5, 1, or 0.5 ng/ml. Particular exemplary EC₅₀ values are also shown in the Examples. For example, CO-1 scFv has shown an EC₅₀ value of as low as 0.05 ng/ml against Jurkat T cells (see FIG. 2).

Preferred antibodies (or binding proteins) of the present invention show reduced binding to normal cells expressing CD47 when compared to tumour cells, for example Jurkat cells. In particular, preferred antibodies (or binding proteins) of the invention, including humanized antibodies (or binding proteins) of the invention, show reduced binding to red blood cells (RBCs), e.g. human red blood cells (RBCs) when compared to tumour cells, for example Jurkat cells.

Thus, in preferred embodiments of the invention, antibodies (or binding proteins) show preferential binding, or greater binding, preferably measurably or significantly greater binding, to cancer cells, for example Jurkat cells, as compared to normal cells, for example RBCs or human RBCs, or to normal human B cells (e.g. B cells from buffy coats of healthy human donors). In other words, limited binding to normal cells, in particular RBCs or human RBCs, or to normal human B cells (e.g. B cells from buffy coats of healthy human donors), is observed.

In some embodiments, antibodies (or binding proteins) of the invention show at least 50, 75, 100, 150, 200, 250, 300, 350, 400, 450, 500, 550, 600, 650, 700, 750 or 800% increased binding to Jurkat cells as compared to human RBCs or to normal human B cells. Put another way, antibodies of the invention show at least 1.5 fold, 2 fold, 3-fold, 4-fold, 5-fold, 6-fold, 7-fold, 8-fold, 9-fold or 10-fold increased binding to Jurkat cells as compared to human RBCs or to normal human B cells.

The preferred EC₅₀ values as described above and elsewhere herein are preferably as determined in an appropriate binding assay carried out under appropriate conditions to enable EC₅₀ values to be measured or determined, e.g. an appropriate flow cytometry based assay, for example as described above or in the Examples section. These assays can be carried out with any appropriate antibody (or binding protein) format. Thus, the exemplary values provided above and elsewhere herein can for example be values as determined when an antibody is in the scFv format, or an alternative format which is monovalent for CD47 (e.g. a Fab format).

Antibodies (or binding proteins) of the invention, including humanized antibodies (or binding proteins) of the invention, are capable of killing tumour cells (e.g. CD47 expressing tumour cells), for example are capable of the direct killing of tumour cells (e.g. CD47 expressing tumour cells). It is believed that this killing takes place by a programmed cell death (PCD) pathway. It is further believed that the PCD pathway is caspase independent.

Such killing is described as direct, which means that it can result in killing of cells by the antibody (or binding protein) itself, for example other entities, e.g. cells, such as immune effector cells, such as macrophages, are not required for the killing to take place. This is in contrast for example to entities which rely on the blocking or inhibition of the CD47-SIRPα interaction, where tumour cell destruction takes place by phagocytosis, which involves the recruitment of other cells, for example macrophages, or other innate immune cells.

The ability of the antibodies (or binding proteins) of the invention to induce such direct cell killing or PCD, in particular of tumour cells, is particularly advantageous. Indeed, many previously described anti-CD47 antibodies do not exhibit this property. The antibodies of the invention have been shown to be particularly effective in inducing this killing, for example are able to kill, or induce PCD of, a significant number/percentage of tumour cells. In addition, they have been shown to demonstrate this effect quickly and/or at a very low concentration. The antibodies of the invention are also capable of inducing this killing (PCD) when they are in a soluble format, as opposed to, for example, when immobilised. Again, this is an advantageous property, as it means that such antibodies can work in solution or in a soluble format which would be the usual and convenient format for therapeutic antibody administration.

PCD can conveniently be measured using an Annexin V/7-ADD assay which would be well known to a person skilled in the art. Indeed, kits are commercially available for carrying out such assays and can be used (e.g. eBioscience™ Annexin V Apoptosis Detection Kit eFluor™ 450 from ThermoFisher, cat.no. 88-8006-74). Cells which are Annexin V positive and 7AAD negative are regarded as early apoptotic cells, whereas cells which are Annexin V positive and 7AAD positive are regarded as late apoptotic cells. Although the numbers/% s of early and late apoptotic cells can be analysed separately, conveniently these two categories of apoptotic cells are added together in order to arrive at an overall determination of the amount of cell death. Such overall PCD can conveniently be expressed as a %.

Antibodies (or binding proteins) of the invention can induce PCD at concentrations of less than 10 μg/ml, 5 μg/ml, 2 μg/ml, 1 μg/ml or 0.5 μg/ml. Further preferred antibodies of the invention can induce PCD at very low concentrations, e.g. at, or less than, 0.1 μg/ml, e.g. from between 0.05 or 0.1 to 1, 2, 5 or 10 μg/ml.

Preferred antibodies of the invention can induce PCD relatively quickly, e.g. after contact times of as low as 30 minutes. Longer contact times, for example 1 h, 2 h or 3 h are also effective to induce PCD. Even longer contact times can also be effective, e.g. 6 h or 12 h. However, clearly it is advantageous if the antibodies are capable of working quickly.

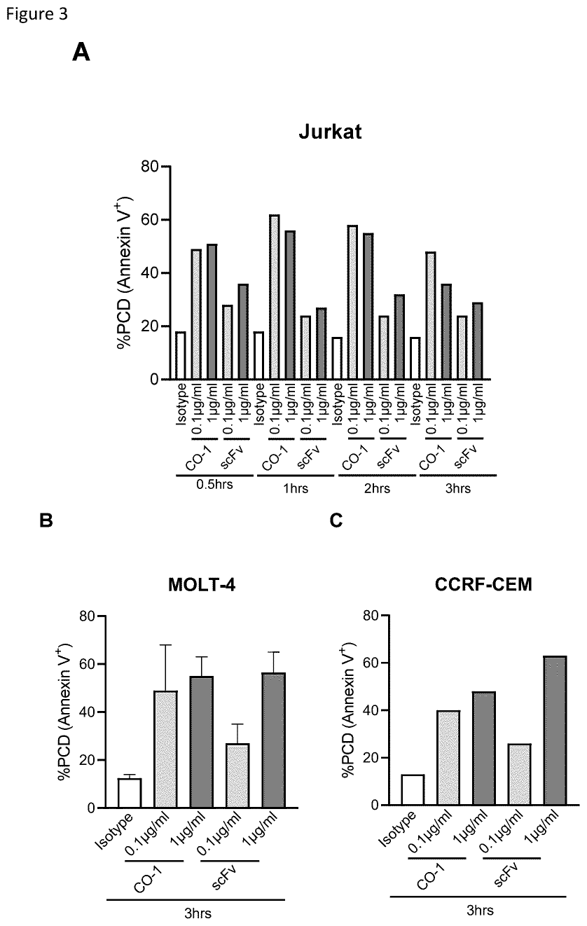

The antibodies (or binding proteins) of the invention are preferably capable of inducing direct cell killing of a wide range of cancer cells, for example cells from haematological cancers and also from solid tumours. Purely by way of example, and not meant to provide an exhaustive list, the antibodies (or binding proteins) of the invention have been shown to be capable of killing haematological cancer cells such as CCRF-CEM, Jurkat, and MOLT-4 (See FIG. 3). The % PCD values will naturally vary depending on the cell type concerned. However, the exemplified antibodies of the invention (including humanized antibodies of the invention), show good % killing levels to many cancer cells, for example levels of at least 10% killing are regularly seen for different types of cancer cells, with levels of at least 15%, 20%, 25%, 30%, 35%, 40%, 45%, 50%, 55%, or 60%, e.g. up to 30%, 35%, 40%, 45%, 50%, 55%, or 60%, being observed for some types of cancer cells. Purely by way of example, antibodies (or binding proteins) of the invention can induce at least, or up to, 20%, 25%, or 30% killing of Jurkat cells (a cancerous T cell line), for example when the cells were exposed for 30 minutes, 1 hour, 2 hours or 3 hours to an scFv antibody of the invention at a concentration of 1 or 0.1 μg/ml. Purely by way of example, antibodies (or binding proteins) of the invention, e.g. humanized antibodies (or binding proteins) of the invention, can induce at least, or up to, 35%, 40%, 45%, 50%, 55%, 60% or 65% killing of Jurkat cells (a cancerous T cell line), for example when the cells were exposed for 30 minutes, 1 hour, 2 hours or 3 hours to an scFv antibody of the invention at a concentration of 1 or 0.1 μg/ml.

Purely by way of further example, antibodies (or binding proteins) of the invention can induce at least, or up to, 40%, 45%, 50%, 55%, or 60% killing of CCRF-CEM cells, for example when the cells were exposed for 3 hours to an scFv antibody of the invention at a concentration of 1 or 0.1 μg/ml.

This killing can be observed when the antibodies are used at 1 μg/ml and as low as 0.1 μg/ml. This direct killing effect can be observed when the antibodies are incubated with cells for 3 hours. However, the antibodies have been shown to also be effective with shorter incubation times than this, in other words the antibodies are fast acting and potent. For example, significant cell death can be observed after an incubation time of 30 minutes up to for example 1, 2 or 3 hours.

The ability of the antibodies or binding proteins of the invention to induce PCD at all, let alone at the above levels, is surprising and advantageous, as these antibodies or binding proteins bind monovalently to CD47 and it has been suggested in the art that divalent binding to CD47 might be important for the induction of PCD.

Methods of calculating % PCD values would be well known to a skilled person. However, conveniently the % PCD values herein can be determined in an in vitro assay, e.g. an AnnexinV/7-AAD assay, by incubating an appropriate cell line with a certain concentration of a test antibody of the invention (e.g. from 0.1 to 1 μg/ml) for a certain length of time (e.g. 30 mins to 12 hours, e.g. approximately 30 mins, 1 hour, 2 hours, 3 hours, 6 hours, or 12 hours), followed by staining with AnnexinV eFluor405 and 7-AAD. The % PCD is the sum of the Annexin V⁺ 7-AAD⁻ cells and the Annexin V⁺ 7-AAD⁺ cells as analysed by flow cytometry.

In some embodiments, antibodies (or binding proteins) of the invention (including humanized antibodies or binding proteins) do not induce significant killing (PCD) of normal human B cells (e.g. B cells from buffy coats of healthy human donors).

Preferred antibodies (or binding proteins) of the invention do not induce haemagglutination (or significant haemagglutination) of red blood cells (RBCs). This is a surprising and advantageous property, especially when combined with the ability to induce PCD of tumour cells as described elsewhere herein. It can be noted that the humanized antibodies of the invention can retain such properties.

The ability to induce haemagglutination can conveniently be measured using an in vitro haemagglutination assay which would be well known to a person skilled in the art. Such assays can conveniently involve bringing increasing concentrations of a test antibody (or controls) into contact with a preparation of RBCs, e.g. human RBCs, e.g. freshly isolated human RBCs, e.g. 2% (v/v) freshly isolated RBCs, and incubating the mixture for a certain amount of time (e.g. 30 to 60 minutes) or until cells have settled in the well or receptacle used (conveniently 96 well plates are used for such assays). A diffuse hazy pattern indicates haemagglutination, while a small punctate circle indicates no haemagglutination.

Preferred antibodies (or binding proteins) of the invention, including humanized antibodies of the invention, do not induce haemagglutination (or significant haemagglutination) of RBCs when used at a concentration of, or at least, or up to 1, 2, 5 or 10 μg/ml. Preferred antibodies of the invention do not induce haemagglutination (or significant haemagglutination) of RBCs when used at a concentration of, or at least, or up to 15, 20, 25, 50, 75 or 100 μg/ml. Although any convenient method of assessing haemagglutination can be used, a suitable and preferred assay is described above and in the Examples section. Thus, the values as described above may be values as, when or if determined in a haemagglutination assay as described above. Particularly preferred methods are described in the Example section herein.

Preferably, the above described abilities and properties are observed at a measurable or significant level and more preferably at a statistically significant level, when compared to appropriate control levels. Appropriate significance levels are discussed elsewhere herein. More preferably, one or more of the above described abilities and properties are observed at a level which is measurably better, or more preferably significantly better (preferably statistically significantly better), when compared to the abilities observed for prior art antibodies.

In any statistical analysis referred to herein, preferably the statistically significant difference over a relevant control or other comparative entity or measurement has a probability value of ≤0.1 or <0.1, preferably ≤0.05 or <0.05. Appropriate methods of determining statistical significance are well known and documented in the art and any of these may be used.

In some embodiments, binding proteins or antibodies of the present invention, including the CO-1scFv antibody, the humanized CO-1 scFv antibodies, and the monovalent scFv-Fc fusion proteins of the invention, have one or more, preferably two or more, or three or more, or four or more, most preferably all, of the functional properties, in particular the preferred functional properties, described herein. Examples of preferred functional properties and further details regarding said properties are described elsewhere herein, and include i) a high affinity for CD47, e.g. when measured by SPR (Biacore), ii) the ability to induce direct killing of tumor cells, e.g. Jurkat cells or CCRF-CEM cells, which can be induced iii) at low concentrations (e.g. at an antibody concentration of 0.1 or 1 μg/ml) and/or iv) after short incubation times (e.g. 30 minutes or 1 hour), v) the ability to not induce haemagglutination (or significant haemagglutination) of red blood cells (RBCs).

Thus, in some embodiments, the present invention provides a binding protein, for example an antibody, comprising one antigen binding domain that binds to CD47, said antigen binding domain comprising a heavy chain variable region that comprises three complementarity determining regions (CDRs), and a light chain variable region that comprises three CDRs, wherein said binding protein or antibody binds monovalently to CD47, and wherein said binding protein or antibody has one or more, preferably two or more, or three or more, or four or more, most preferably all, of the functional properties, in particular the preferred functional properties, described herein, e.g. one or more etc., of the properties (i) to (v) above. In some embodiments said binding protein or antibody also has the ability to induce phagocytosis of tumour cells.

In some embodiments, the functional property (ii) is preferred, optionally together with one or more of the other properties listed above. In some embodiments, the functional property (ii) is preferred, optionally together with one or more, two or more, three or more, or most preferably all, of the properties (i), (iii), (iv) and (v) listed above, or one or more, two or more, or most preferably all, of the properties (iii), (iv) and (v) listed above. In some such embodiments said binding protein or antibody also has the ability to not induce phagocytosis of tumour cells.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”.

In addition, where the terms “comprise”, “comprises”, “has” or “having”, or other equivalent terms are used herein, then in some more specific embodiments, for example in the definition of the CDR or FR sequences herein, these terms include the term “consists of” or “consists essentially of”, or other equivalent terms.

Nucleic acid molecules (e.g. one or more nucleic acid molecules) comprising nucleotide sequences that encode the binding proteins or antibodies or immunoconjugates of the present invention as defined herein, or nucleic acid molecules substantially homologous thereto, form yet further aspects of the invention.

Preferred nucleic acid molecules are those encoding an antibody of the present invention as described elsewhere herein which can bind monovalently to CD47, e.g. an antibody of the invention with CDR and optionally FR and other regions as defined in Table A or B, or antibodies with sequences substantially homologous thereto.

Preferred nucleic acid molecules are those encoding an antibody of the present invention which can bind monovalently to CD47 (e.g., comprising nucleic acid sequences encoding SEQ ID NO:3 and/or SEQ ID NO:4, such as SEQ ID NO:1 and/or SEQ ID NO:2, respectively).

Other preferred nucleic acid molecules comprise sequences that encode an scFv format, or an alternative format which is monovalent for CD47 (e.g. a Fab format), of the antibodies of the invention, for example those as described in Table A, herein (heavy variable chains and light chains). Thus, preferred nucleic acid molecules are those encoding the scFv of SEQ ID NO: 21 or which encode an alternative format which is monovalent for CD47 and for example encodes the VH domain of SEQ ID NO:3 and/or the VL domain of SEQ ID NO:4. Other preferred nucleic acid molecules comprise sequences that encode humanized antibodies of the invention, for example encode those antibodies as described in Table B (heavy chains and light chains).

Thus, preferred nucleic acid molecules are those encoding a heavy chain of an antibody of the present invention (e.g. those encoding SEQ ID NOs: 22, 23, 24, 25 or 26) and/or those encoding a light chain of an antibody of the present invention (e.g. those encoding SEQ ID NO: 27, 28 or 29). Other preferred nucleic acid molecules comprise sequences that encode the monovalent scFv-Fc fusion proteins of the invention (e.g. CO201-scFv-Fc-mono).

The term “substantially homologous” as used herein in connection with an amino acid or nucleic acid sequence includes sequences having at least 60%, 65%, 70% or 75%, preferably at least 80%, and even more preferably at least 85%, 90%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions, or deletions) to the sequences of the invention. At the amino acid level preferred substantially homologous sequences contain up to 6, e.g. only 1, 2, 3, 4, 5 or 6, for example 1, 2, 3, 4 or 5, preferably 1, 2, 3 or 4, preferably 1, 2 or 3, more preferably 1 or 2, altered amino acids, in one or more of the framework regions and/or one or more of the CDRs making up the sequences of the invention. In addition, at the amino acid level, preferred substantially homologous sequences contain up to 6, e.g. only 1, 2, 3, 4, 5 or 6, for example 1, 2, 3, 4 or 5, preferably 1, 2, 3 or 4, preferably 1, 2 or 3, more preferably 1 or 2, altered amino acids, in the combined framework regions (e.g. the four framework regions), and/or the combined CDRs (e.g. the three CDR regions) making up the VL or VH domains of the antibodies of the invention. In addition, at the amino acid level, preferred substantially homologous sequences contain up to 6, e.g. only 1, 2, 3, 4, 5 or 6, for example 1, 2, 3, 4 or 5, preferably 1, 2, 3 or 4, preferably 1, 2 or 3, more preferably 1 or 2, altered amino acids, in the VH domain and/or the VL domain of the antibodies of the invention. Said alterations can be with conservative or non-conservative amino acids, or a mixture thereof. Preferably said alterations are substitutions, preferably conservative amino acid substitutions.

In certain embodiments, if a given starting sequence is relatively short (e.g. five amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR1 sequence in accordance with the present invention, e.g. a starting VH CDR1 sequence which in some embodiments may be five amino acid residues in length, preferably has 1 or 2 (more preferably 1) altered amino acids in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 55%, 60%, 65%, 70%, 75%, 80%, 85%, 90%, 95%, 96%, 97%, 98% or 99%.

Routine methods in the art such as alanine scanning mutagenesis and/or deep mutational scanning (which aims to perform all possible mono-substitutions on all selected residues within a given protein sequence) and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

Once identified, the addition, deletion, substitution or insertion of one or more amino acids in the amino acid sequence of a parent antibody to form a new antibody, wherein said parent antibody is one of the antibodies of the invention as defined elsewhere herein, and testing the resulting new antibody to identify antibodies that bind to CD47 in accordance with the invention can be carried out using techniques which are routine in the art. Such methods can be used to form multiple new antibodies that can all be tested for their ability to bind CD47. Preferably said addition, deletion, substitution or insertion of one or more amino acids takes place in one or more of the CDR domains.

For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way. Testing the ability of one or more of the modified antibodies/binding proteins to bind to CD47 can be carried out by any appropriate method, which are well known and described in the art. Suitable methods are also described elsewhere herein and in the Examples section.

New antibodies produced, obtained or obtainable by these methods form a yet further aspect of the invention.

The term “substantially homologous” also includes modifications or chemical equivalents of the amino acid and nucleotide sequences of the antibodies of the present invention that perform substantially the same function as the proteins or nucleic acid molecules of the antibodies of the invention in substantially the same way. For example, any substantially homologous antibody should retain the ability to bind monovalently to CD47 as described above. Preferably, any substantially homologous antibody should retain one or more (or all) of the functional capabilities of the starting antibody.

Substantially homologous sequences of proteins of the invention include, without limitation, conservative amino acid substitutions, or for example alterations that do not affect the VH, VL or CDR domains of the antibodies, e.g. antibodies where tag sequences, toxins or other components are added that do not contribute to the monovalent binding of CD47 antigen.

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). In other examples, families of amino acid residues can be grouped based on hydrophobic side groups or hydrophilic side groups.

Homology or sequence identity may be assessed by any convenient method. However, for determining the degree of homology or identity between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having at least 55%, 60%, 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

Preferably, any substantially homologous antibody of the invention should retain the ability to specifically bind to the same epitope of CD47 as recognized by the starting antibody in question, for example, the same epitope recognized by the CDR domains of one or more of the antibodies of the invention or the VH and VL domains of one or more of the antibodies of the invention as described herein, e.g. bind to the same epitope as one or more of the various antibodies of the invention (e.g. the CD47 antibody as shown in Table A or B).

Binding to the same epitope can be readily tested by methods well known and described in the art, e.g. using epitope mapping assays, e.g. by analysis of the crystal structure of the antigen-antibody complex, or by mutational studies of individual residues (e.g. using alanine scanning and/or deep mutational scanning, DMS, for example yeast display in combination with DMS, see for example as described in Sierocki et al., 2021, PLoS Negl Trop Dis., 15 (3): e0009231; see also Van Blarcom et al., 2015, JMB, 427:6(B):1513-1534, and Medina-Cucurella and Whitehead, 2018, Methods Mol. Biol., 1764:101-121). In some embodiments DMS, and in particular yeast display in combination with DMS, is preferred as a method for determining the epitope. Any of the above such analysis to determine the epitope can be used in conjunction with binding assays, e.g. a competition assay, for example as part of an initial screening. Thus, antibodies which bind to the same epitope as one or more of the various antibodies of the invention, for example as assessed or determined by analysis of the crystal structure of the antigen-antibody complex, or by mutational studies of individual residues (e.g. using alanine scanning and/or deep mutational scanning, DMS, for example yeast display in combination with DMS), form yet further aspects of the invention. In a preferred embodiment, DMS, for example yeast display in combination with DMS, is used to assess or determine the epitope. Retention of other functional properties, in particular binding affinity, can also readily be tested by methods well known and described in the art or herein.

Thus, a person skilled in the art will appreciate that such methods can be used to test whether any antibodies, for example “substantially homologous” antibodies, have the same binding specificities, e.g. bind to the same epitope, or with the same or equivalent affinity, as the antibodies and antibody fragments of the invention. For example, analysis of the crystal structure of the antigen-antibody complex, or mutational studies of individual residues (e.g. using alanine scanning and/or deep mutational scanning, DMS, for example yeast display in combination with DMS), can readily be used to assess whether antibodies, for example “substantially homologous” antibodies, can bind to the same epitope of CD47, optionally supplemented with binding assays such as competition assays as described elsewhere herein. SPR assays, such as BIAcore assays as described elsewhere herein, could also readily be used to establish whether antibodies, for example “substantially homologous” antibodies, can bind to CD47, and optionally the affinity, e.g. K_{D}, of such binding. The skilled person will be aware of other suitable methods and variations.

As outlined below, a competition binding assay can be used as an initial or supplementary assay to an epitope mapping assay to test whether antibodies, for example “substantially homologous” antibodies retain the ability to specifically bind to the same (or substantially the same) epitope of CD47 as recognized by one or more of the antibodies of the invention, e.g. as shown in the sequence Tables herein, or have the ability to compete with one or more of the various antibodies of the invention as shown in the sequence Tables herein. The method described below is only one example of a suitable competition assay. The skilled person will be aware of other suitable methods and variations.

An exemplary competition assay involves assessing the binding of various effective concentrations of an antibody of the invention to CD47 in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to CD47) is evidence of binding to the same or substantially the same epitope. Preferably, the test antibody significantly reduces the amount of antibody of the invention that binds to CD47. Preferably, the test antibody reduces the amount of antibody of the invention that binds to CD47 by at least about 95%. ELISA or flow cytometry assays may be used for assessing inhibition of binding in such a competition assay but other suitable techniques would be well known to a person skilled in the art.

Such antibodies (or binding proteins) which have the ability to bind (or specifically bind) to the same (or substantially the same) epitope of CD47 as recognized by the antibodies of the invention (e.g. the antibody as shown in Table A), and wherein said binding protein or antibody binds monovalently to CD47, are further embodiments of the present invention. Such antibodies (or binding proteins) optionally also have the ability to compete with one or more of the various antibodies of the invention (e.g. the antibody as shown in Table A) for binding to CD47.

Thus, a yet further aspect of the invention provides an antibody (or binding protein), which binds monovalently to (or specifically binds monovalently to) CD47 and which has the ability to bind to the same (or substantially the same) epitope as the CO-1 scFv (Table A) antibody, i.e. an antibody comprising the VL of SEQ ID NO:4 and the VH of SEQ ID NO:3, as described herein, or the ability to bind to the same (or substantially the same) epitope as an antibody comprising the same CDRs as the CO-1 scFv (Table A) antibody, i.e. an antibody comprising VL CDR sequences of SEQ ID NOs: 8, 9 and 10 and VH CDR sequences of SEQ ID NOs: 5, 6 and 7, for binding to CD47. Such antibodies (or binding proteins) optionally also have the ability to compete with one or more of the various antibodies of the invention (e.g. the antibody as shown in Table A) for binding to CD47. Other features and properties of other aspects of the invention apply, mutatis mutandis, to this aspect of the invention.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to CD47. Preferably, the competing antibody can bind to the same epitope as the reference antibody. Alternatively viewed, the competing antibody preferably has the same epitope specificity as the reference antibody.

“Reference antibodies” as used herein are antibodies which can bind to CD47 in accordance with the invention which preferably have a VH and a VL domain as defined herein in Table A, more preferably have a VH domain comprising SEQ ID NO: 3 and a VL domain comprising SEQ ID NO: 4 (or the relevant three CDR sequences of said sequences) as outlined in Table A, or which have a humanized VH and/or VL domain as shown in Table B.

The identification of one or more antibodies that bind to the same epitope, and optionally competing antibodies, is a straightforward technical matter now that reference antibodies such as those outlined in the sequence Tables herein have been provided. Epitope mapping can be performed using standard techniques, some of which are outlined elsewhere herein. Such epitope mapping can also be supplemented with competition assays, e.g. as an initial or supplementary screening step, which can be performed using standard techniques, some of which are outlined elsewhere herein. In this regard for example, CD47 antibodies can be generated by immunization protocols using CD47 antigen or preferably cells expressing or over-expressing CD47 antigen as an immunogen, after which such CD47 antibodies can readily be screened, for example using methods as described herein, to identify those that bind to the same epitope as the reference antibodies of the invention. Alternatively, substantially homologous sequences derived from antibodies with the sequences shown in Table A or Table B can be screened in this way.

Epitope mapping using DMS, and in particular yeast display in combination with DMS, has indeed been carried out for the CO-1 antibodies of the invention.

Thus, in another aspect, the present invention provides a binding protein, for example an antibody, for example a binding protein or antibody as described elsewhere herein, comprising one antigen binding domain that binds to CD47, said antigen binding domain comprising a heavy chain variable region that comprises three complementarity determining regions (CDRs), and a light chain variable region that comprises three CDRs, wherein said antigen binding domain binds to (or is capable of binding to, or specifically binding to) Q19, N45, T120, R121, E122 and G123 within CD47 as defined by SEQ ID NO:19, and wherein said binding protein or antibody binds monovalently to CD47. In other embodiments, said antigen binding domain binds to Q19, L20, N45, T120, R121, E122 and G123 within CD47 as defined by SEQ ID NO:19.

Viewed alternatively, said antigen binding domain binds to an epitope comprising or consisting of Q19, N45, T120, R121, E122 and G123 within CD47 as defined by SEQ ID NO:19. In other embodiments, said antigen binding domain binds to an epitope comprising or consisting of Q19, L20, N45, T120, R121, E122 and G123 within CD47 as defined by SEQ ID NO:19.

In some embodiments, said antigen binding domain does not bind to, or said epitope does not comprise, one or more, two or more, three or more, and preferably all, of T117, E118, E115 and L21. In some embodiments said antigen binding domain does not bind to, or said epitope does not comprise, T117, or T117 and E118, or T117, E118 and E115, or T117, E118, E115 and L21, within CD47 as defined by SEQ ID NO:19.

In some embodiments, said antigen binding domain does not bind to, or said epitope does not comprise, one or more, two or more, three or more, and preferably all, of V54 to A84, M46, or L119, in particular L119, within CD47 as defined by SEQ ID NO:19.

In some embodiments, said antigen binding domain does not bind to, or said epitope does not comprise, one or more, two or more, three or more, four or more, and preferably all, of E115 to L119, in particular T117 to L119, in particular T117 and/or L119, within CD47 as defined by SEQ ID NO:19.

In some embodiments, said antigen binding domain does not bind to, or said epitope does not comprise, one or more, and preferably both, of T117 and E124, within CD47 as defined by SEQ ID NO:19.

In some embodiments, said antigen binding domain does not bind to, or said epitope does not comprise, one or more, two or more, three or more, and preferably all, of L21, E47, E115 and E118, within CD47 as defined by SEQ ID NO:19.

In some embodiments, said antigen binding domain does not bind to, or said epitope does not comprise, one or more, two or more, three or more, four or more, and preferably all, of M46, Y55, K57, D69, and L119, within CD47 as defined by SEQ ID NO:19.

In some embodiments, the binding of said antigen binding domain to CD47 as defined by SEQ ID NO:19, is not dependent on G70. For example, in such embodiments, mutation of G70 to another amino acid residue does not affect, or does not significantly affect, the binding the binding of the antigen binding domains or antibodies of the invention to CD47, or the binding is maintained. In some embodiments, said antigen binding domain does not bind to, or said epitope does not comprise the residue G70 within CD47 as defined by SEQ ID NO:19.

In preferred embodiments, binding proteins or antibodies which bind to the epitopes or residues as outlined above, are as assessed or determined by analysis of the crystal structure of the antigen-antibody complex, or by mutational studies of individual residues (e.g. using alanine scanning and/or deep mutational scanning, DMS, for example yeast display in combination with DMS). In a preferred embodiment, binding to such epitopes or residues is as assessed or determined by DMS, for example yeast display in combination with DMS.

In preferred embodiments, the binding proteins or antibodies that bind to the CD47 epitope as defined herein, have a binding affinity as described elsewhere herein. Thus, the present invention provides a binding protein, for example an antibody, comprising one antigen binding domain that binds to the CD47 epitope as defined herein, said antigen binding domain comprising a heavy chain variable region that comprises three complementarity determining regions (CDRs), and a light chain variable region that comprises three CDRs, wherein said binding protein or antibody binds monovalently to CD47, and wherein said binding protein or antibody has a binding affinity as defined elsewhere herein. Thus, preferably, such antibodies or binding proteins, for example when in scFv format or monovalent scFv-Fc format, or an alternative format which is monovalent for CD47 (e.g. a Fab format), have a binding affinity for CD47 (e.g. human CD47, e.g. with SEQ ID NO:19) that is or corresponds to a K_{D} of less than 750 pM, preferably less than 700 pM, less than 650 pM, less than 600 pM or less than 550 pM, more preferably less than 500, 450, 400, 350, 300, 250, 200, 150, 125, 100, 75, 50 or 40 pM.

The terms “antibody” and “immunoglobulin”, as used herein, refer broadly to any immunological binding agent that comprises an antigen binding domain, including polyclonal and monoclonal antibodies. Monoclonal antibodies are preferred. However, binding proteins and antibodies of the invention have a structure or format such that they bind monovalently to CD47, e.g. can be antibody fragments that bind monovalently to CD47.

The term “heavy chain complementarity determining region” (“heavy chain CDR”) as used herein refers to regions of hypervariability within the heavy chain variable region (VH domain) of an antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (VH domain) as used herein refers to the variable region of a heavy chain of an antibody molecule.

The term “light chain complementarity determining region” (“light chain CDR”) as used herein refers to regions of hypervariability within the light chain variable region (VL domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (V_{L} domain) as used herein refers to the variable region of a light chain of an antibody molecule.

As described elsewhere herein, binding proteins and antibodies of the invention have a structure or format such that they bind monovalently to CD47. Any appropriate monovalent format may be used, e.g. any format of antibody or antibody fragment which contains only one antigen binding domain that can bind to CD47. Exemplary and preferred formats or fragments are scFv antibodies or Fab or Fab′ fragments. Other exemplary monovalent formats or fragments include sdAb (nanobody), unibody, monovalent scFv-Fc fusion, and monovalent IgG (half-antibody). As described elsewhere herein a particularly preferred format is monovalent scFv-Fc.

The antibodies or binding proteins can be produced naturally or can be wholly or partially synthetically produced.

The antigen binding domains of the antibodies or binding proteins of the invention generally comprise an antibody light chain variable region (V_{L}) that comprises three CDR domains and an antibody heavy chain variable region (V_{H}) that comprises three CDR domains.

However, it is well documented in the art that the presence of three CDRs from the light chain variable domain and three CDRs from the heavy chain variable domain of an antibody is not always necessary for antigen binding. Thus, constructs smaller than the above classical antigen binding domain are known to be effective.

For example, camelid VHH antibodies and other single domain antibodies comprising VH domains alone show that these domains can bind to antigen with acceptably high affinities. Thus, three CDRs (or even a single CDR) can effectively bind antigen and form an antigen binding domain.

Thus, although preferred antigen binding domains in the antibodies of the invention might comprise six CDR regions (three from a light chain and three from a heavy chain), antibodies with antigen binding domains with fewer than six CDR regions (e.g. 3 CDR regions) are encompassed by the invention. Antibodies with antigen binding domains with CDRs from only the heavy chain or light chain are also contemplated.

Preferred light chain CDR regions for use in conjunction with the specified heavy chain CDR regions to form the antigen binding domain are described elsewhere herein. However, other light chain variable regions that comprise three CDRs for use in conjunction with the heavy chain variable regions of the invention are also contemplated. Appropriate light chain variable regions which can be used in combination with the heavy chain variable regions of the invention and which give rise to an antibody which binds monovalently to CD47 in accordance with the invention can be readily identified by a person skilled in the art.

For example, a heavy chain variable region of the invention can be combined with a single light chain variable region or a repertoire of light chain variable regions and the resulting antibodies tested for binding to CD47.

If desired, similar methods could be used to identify alternative heavy chain variable regions for use in combination with preferred light chain variable regions of the invention.

The antibody, binding protein and nucleic acid molecules of the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to nucleic acid molecules or sequences and proteins or polypeptides, e.g. antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

Thus, when used in connection with a protein or polypeptide molecule such as light chain CDRs 1, 2 and 3, heavy chain CDRs 1, 2 and 3, light chain variable regions, heavy chain variable regions, and binding proteins or antibodies of the invention, the term “isolated” or “purified” typically refers to a protein substantially free of cellular material or other proteins from the source from which it is derived. In some embodiments, particularly where the protein is to be administered to humans or animals, such isolated or purified proteins are substantially free of culture medium when produced by recombinant techniques, or chemical precursors or other chemicals when chemically synthesized.

It can be noted that in embodiments the antibodies etc., of the invention do not occur in nature and are, in that respect, man-made constructs in that they do not correspond to molecules that occur naturally. For example, preferred antibodies can be engineered or recombinantly produced, or are experimentally induced to be produced in an animal species, e.g. by immunization. In other words in embodiments the antibodies, etc., of the invention are non-native or not naturally occurring.

A person skilled in the art will appreciate that the proteins and polypeptides of the invention, such as the heavy and light chain CDRs, the heavy and light chain variable regions, antibodies and antibody fragments, may be prepared in any of several ways well known and described in the art, but are most preferably prepared using recombinant methods.

Nucleic acid fragments encoding the heavy and/or light chain regions of the antibodies of the invention, as appropriate, can be derived or produced by any appropriate method, e.g. by cloning or synthesis.

Once nucleic acid fragments encoding the heavy and/or light chain regions of the antibodies of the invention have been obtained, these fragments can be further manipulated by standard recombinant DNA techniques. Typically, or as part of this further manipulation procedure, the nucleic acid fragments encoding the antibody molecules of the invention are generally incorporated into one or more appropriate expression vectors in order to facilitate production or manipulation of the antibodies of the invention.

Possible expression vectors include but are not limited to cosmids, plasmids, or modified viruses (e.g. replication defective retroviruses, adenoviruses and adeno-associated viruses), so long as the vector is compatible with the host cell used. Transposons can also be used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates an expression vector, e.g. a recombinant expression vector, containing or comprising a nucleic acid molecule of the invention, or a fragment thereof, and the necessary regulatory sequences for the transcription and translation of the protein sequence encoded by the nucleic acid molecule of the invention.

Expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells, as will be well known to a person skilled in the art. For example, the proteins of the invention may be expressed in yeast cells or mammalian cells, for example HEK or CHO cells. In addition, if appropriate, the proteins of the invention may be expressed in prokaryotic cells, such as Escherichia coli (E. coli). Cell-free expression systems might also be used.

The proteins of the invention may also be prepared by chemical synthesis using techniques well known in the chemistry of proteins such as solid phase synthesis.

A yet further aspect provides an expression construct or expression vector or expression system (e.g. a viral or bacterial or other expression construct, vector or system), e.g. one or more expression constructs or expression vectors, comprising one or more of the nucleic acid fragments or segments or molecules of the invention. Preferably the expression constructs or vectors or systems are recombinant. Preferably said constructs or vectors or systems further comprise the necessary regulatory sequences for the transcription and translation of the protein sequence encoded by the nucleic acid molecule of the invention.

A yet further aspect provides a host cell (e.g. a mammalian or bacterial or yeast host cell) or virus, e.g. one or more host cells or viruses, comprising one or more expression constructs or expression vectors of the invention. Also provided are host cells (e.g. a mammalian or bacterial or yeast host cell) or viruses, e.g. one or more host cells or viruses, comprising one or more of the nucleic acid molecules of the invention. A host cell (e.g. a mammalian host cell or bacterial host cell, or yeast host cell) or virus expressing an antibody (or binding protein) of the invention forms a yet further aspect.

A yet further aspect of the invention provides a method of producing (or manufacturing) an antibody (or binding protein) of the present invention comprising a step of culturing the host cells of the invention. Preferred methods comprise the steps of (i) culturing a host cell comprising one or more of the expression vectors or one or more of the nucleic acid sequences of the invention under conditions suitable for the expression of the encoded antibody or binding protein; and optionally (ii) isolating or obtaining the antibody or binding protein from the host cell or from the growth medium/supernatant. Such methods of production (or manufacture) may also comprise a step of purification of the antibody or protein product and/or formulating the antibody or product into a composition, e.g. a pharmaceutical composition, including at least one additional component, such as a pharmaceutically acceptable carrier or excipient or diluent.

In embodiments when the antibody or binding protein of the invention is made up of more than one polypeptide chain (e.g. Fab formats or monovalent scFv-Fc fusion proteins), then all the polypeptides are preferably expressed in the host cell, either from the same or a different expression vector, so that the complete proteins, e.g. antibody proteins of the invention, can assemble in the host cell and be isolated or purified therefrom.

In another aspect, the invention provides a method of binding CD47, comprising contacting a composition comprising CD47 with an antibody or binding protein of the invention.

In yet another aspect, the invention provides a method of detecting CD47, comprising contacting a composition suspected of containing CD47 with an antibody or binding protein of the invention, under conditions effective to allow the formation of CD47/antibody complexes and detecting the complexes so formed.

One therapeutic (or diagnostic) approach is the use of antibodies that can target specific antigens expressed on cancer cells, that are not expressed or are expressed at a lower level on normal cells. These target antigens, of which CD47 is an example, can be exploited using antibodies to specifically kill antigen-bearing tumour cells by a variety of mechanisms including by delivering immuno- or radio labelled conjugates that, when delivered to the antigen-bearing cell, specifically kill the target cell. Such targeting can also be used for diagnosis.

The invention thus also provides a range of conjugated antibodies and binding proteins (immunoconjugates) in which the anti-CD47 antibody (or binding protein) of the invention is operatively attached to at least one other therapeutic or diagnostic agent. The term “immunoconjugate” is broadly used to define the operative association of the antibody (or binding protein) with another effective agent (e.g. therapeutic or diagnostic agent) and is not intended to refer solely to any type of operative association, and is particularly not limited to chemical “conjugation”. Fusion proteins, e.g. recombinant fusion proteins, are particularly contemplated. So long as the delivery or targeting agent (the anti-CD47 component) is able to bind to the target and the therapeutic or diagnostic agent is sufficiently functional upon delivery, the mode of attachment will be suitable.

For example, in some preferred embodiments, antibodies (or binding proteins) of the invention are part of an immunotoxin or are used (e.g. used therapeutically) as part of immunotoxins wherein the antibody is itself operatively associated or combined with a toxic agent (e.g. a chemotherapeutic agent or toxin or a radioactive material such as a radiotracer, e.g. for use in radioimmunotherapy). The operative attachment includes all forms of direct and indirect attachment as described herein and known in the art.

Suitable chemotherapeutic agents or toxins are well known and described in the art, for example cytotoxic proteins derived from bacteria or plants can be used. The toxin should be capable of killing target cells once it has been taken up into said cells. Thus, preferred immunoconjugates of the invention are immunotoxins comprising an antibody of the invention linked or otherwise conjugated to a toxin (also sometimes referred to as antibody drug conjugates, ADCs). In preferred immunoconjugates, active ingredients such as radionuclides, toxins (for example, the diphtheria toxin), or other cytostatic agents can be bonded (conjugated) or otherwise linked to the corresponding antibodies.

Immunoconjugates involving conjugation with RNA molecules such as siRNA, or DNA molecules can also be used.

In some embodiments, antibodies of the invention are used (e.g. used therapeutically) in their “naked” unconjugated form.

Yet further aspects are methods of diagnosis or imaging of a subject comprising the administration of an appropriate amount of an antibody or binding protein of the invention as defined herein to the subject and detecting the presence and/or amount and/or the location of the antibody or binding protein of the invention in the subject.

Appropriate diseases to be imaged or diagnosed in accordance with the present invention are cancers, e.g. as described elsewhere herein in connection with disease treatments.

In one embodiment, the invention provides a method of diagnosing cancer in a mammal comprising the step of:
(a) contacting a test sample taken from said mammal with one or more of the antibodies or binding proteins of the invention.

In a further embodiment, the invention provides a method of diagnosing cancer in a mammal comprising the steps of:
(a) contacting a test sample taken from said mammal with one or more of the antibodies (or binding proteins) of the invention;
(b) measuring the presence and/or amount and/or location of antibody-antigen complex in the test sample; and, optionally
(c) comparing the presence and/or amount of antibody-antigen complex in the test sample to a control.

In the above methods, said contacting step is carried out under conditions that permit the formation of an antibody-antigen complex. Appropriate conditions can readily be determined by a person skilled in the art.

In the above methods any appropriate test sample may be used, for example biopsy cells, tissues or organs suspected of being affected by disease or histological sections.

In certain of the above methods, the presence of any amount of antibody-antigen complex in the test sample would be indicative of the presence of disease. Preferably, for a positive diagnosis to be made, the amount of antibody-antigen complex in the test sample is greater than, preferably significantly greater than, the amount found in an appropriate control sample. More preferably, the significantly greater levels are statistically significant, preferably with a probability value of <0.05. Appropriate methods of determining statistical significance are well known and documented in the art and any of these may be used.

Appropriate control samples could be readily chosen by a person skilled in the art, for example, in the case of diagnosis of a particular disease, an appropriate control would be a sample from a subject that did not have that disease. Appropriate control “values” could also be readily determined without running a control “sample” in every test, e.g. by reference to the range for normal subjects known in the art.

For use in the diagnostic or imaging applications, the antibodies (or binding proteins) of the invention may be labeled with a detectable marker such as a radioisotope, such as ³H, ¹⁴C, ³²P, ³⁵S, ¹²³I, ¹²⁵I, ¹³¹I; a radioactive emitter (e.g. α, β or γ emitters); a fluorescent (fluorophore) or chemiluminescent (chromophore) compound, such as fluorescein isothiocyanate, rhodamine, luciferin or Europium; an enzyme, such as alkaline phosphatase, beta-galactosidase or horseradish peroxidase; an imaging agent; or a metal ion; or a chemical moiety such as biotin which may be detected by binding to a specific cognate detectable moiety, e.g. labelled avidin/streptavidin. Methods of attaching a label to a binding protein, such as an antibody, are known in the art. Such detectable markers allow the presence, amount or location of binding protein-antigen complexes in the test sample to be examined.

Preferred detectable markers for in vivo use include an X-ray detectable compound, such as bismuth (III), gold (III), lanthanum (III) or lead (II); a radioactive ion, such as copper⁶⁷, gallium⁶⁷, gallium⁶⁸, indium¹¹¹, indium¹¹³, iodine¹²³, iodine¹²⁵, iodine¹³¹, mercury¹⁹⁷, mercury²⁰³, rhenium¹⁸⁶, rhenium¹⁸⁸, rubidium⁹⁷, rubidium¹⁰³, technetium^{99m} or yttrium⁹⁰; a nuclear magnetic spin-resonance isotope, such as cobalt (II), copper (II), chromium (III), dysprosium (III), erbium (III), gadolinium (III), holmium (III), iron (II), iron (III), manganese (II), neodymium (III), nickel (II), samarium (III), terbium (III), vanadium (II) or ytterbium (III); or rhodamine or fluorescein.

The invention also includes diagnostic or imaging agents comprising the antibodies of the invention attached to a label or detectable marker that produces a detectable signal, directly or indirectly. Appropriate labels or detectable markers are described elsewhere herein.

In one embodiment the method of diagnosing cancer is an in vitro method.

In one embodiment the method of diagnosing cancer is an in vivo method.

Alternatively viewed, the present invention provides a method for screening for cancer in a subject.

Compositions comprising at least a first antibody (or binding protein) or immunoconjugate of the invention, or at least a first nucleic acid molecule or expression vector of the invention, or at least a first host cell of the invention, constitute further aspects of the present invention. Formulations (compositions) comprising one or more antibodies, etc., of the invention, optionally in admixture with a suitable diluent, carrier or excipient constitute a preferred embodiment of the present invention. Such formulations may be for pharmaceutical use, and thus compositions of the invention are preferably pharmaceutically acceptable or otherwise acceptable for administration to human or non-human animals, but in particular humans. Suitable diluents, excipients and carriers are known to the skilled man.

Any appropriate mode of administration can be used. The compositions according to the invention may be presented, for example, in a form suitable for oral, nasal, parenteral (e.g. intravenous, intraperitoneal, subcutaneous, intradermal, intramuscular), topical or rectal administration, or for mucosal delivery, and any of these modes of administration, or indeed any other appropriate mode of administration, can be used. In a preferred embodiment, compositions according to the invention are presented in a form suitable for intravenous administration. In some embodiments, compositions according to the invention are presented in a form suitable for intraperitoneal (i.p.) administration. In some embodiments, compositions according to the invention are presented in a form suitable for injection directly into a tumour (intratumoral).

The active compounds (e.g. the antibodies of the invention) as defined herein may be presented in the conventional pharmacological forms of administration, such as tablets, coated tablets, nasal sprays, solutions, emulsions, liposomes, exosomes, powders, capsules or sustained release forms. Conventional pharmaceutical excipients as well as the usual methods of production may be employed for the preparation of these forms. In addition, nucleic acids or nucleic acid based vectors, e.g. mRNA based vectors, or virus-based vectors, may be used for administration of the active compounds of the invention, e.g. by encoding the antibodies or binding proteins of the invention.

Injection solutions may, for example, be produced in the conventional manner, such as by the addition of preservation agents, such as p-hydroxybenzoates, or stabilizers, such as EDTA. The solutions may then be filled into injection vials or ampoules.

The pharmaceutical compositions (formulations) of the present invention are preferably administered parenterally. Intravenous administration is preferred. In some embodiments, administration is intraperitoneal (i.p.) administration. In some embodiments, administration is by injection into a tumour. Parenteral administration may be performed by subcutaneous, intramuscular, intraperitoneal or intravenous injection by means of a syringe. Alternatively, parenteral administration can be performed by means of an infusion pump. A further option is a composition which may be a powder or a liquid for the administration of the antibody in the form of a nasal or pulmonal spray. As a still further option, the antibodies of the invention can also be administered transdermally, e.g. from a patch, optionally an iontophoretic patch, or transmucosally, e.g. bucally.

Suitable dosage units can be determined by a person skilled in the art.

A further aspect of the present invention provides the anti-CD47 antibodies (or binding proteins) or immunoconjugates defined herein for use in therapy, in particular for use in the treatment or prevention of cancer. In other embodiments, the nucleic acid molecules, expression vectors, host cells or viruses of the invention can also be used in the therapeutic methods described herein.

In accordance with the present invention the antibodies may target CD47 positive cells, e.g. tumour cells.

In one embodiment, solid tumours are treated.

In one embodiment, hematological or blood cancers are treated.

In some embodiments, a tumour or cancer that is characterized by expressing or over-expressing CD47 (e.g. on its surface) is treated.

Thus, a further aspect of the present invention provides anti-CD47 antibodies (or binding proteins) as defined herein for use in the treatment or prevention of a cancer or tumour that is characterized by (or associated with) CD47 expression or over-expression, for example a cancer or tumour characterized by undesired, inappropriate, aberrant, increased or excessive CD47 expression.

In some embodiments, the cancer is characterised by (or associated with) CD47 signalling (e.g. aberrant or inappropriate or undesired CD47 signalling).

In some embodiments the antibodies (or binding proteins) of the present invention can induce direct killing of CD47 expressing cancer or tumor cells.

Preferred cancers to be treated in accordance with the present invention include lung cancer, e.g. non-small cell lung cancer (NSCLC, e.g. squamous NSCLC), small cell lung cancer (e.g extensive stage disease small cell lung cancer), melanoma (e.g. metastatic melanoma such as BRAF negative metastatic melanoma, or multiple melanoma), lymphoma (e.g. acute T-cell lymphoma, Hodgkin lymphoma, non-Hodgkin lymphoma, e.g. B cell or T cell non-Hodgkin lymphoma or Burkitt's lymphoma, or chronic lymphocytic lymphoma), leukemia (e.g acute myeloid leukemia, acute lymphoblastic leukemia or myelodysplastic syndrome), renal cell cancer (RCC, e.g clear cell renal cancer), colorectal cancer, urothelial bladder cancer, urethral cancer, head and neck cancer (e.g. recurrent or metastatic head and neck squamous cell cancer), breast cancer (e.g. metastatic HER-2 negative breast cancer) advanced liver cancer, brain cancer (e.g. glioblastoma or astrocytoma), stomach cancer, oesophageal cancer, pancreatic carcinoma, adenocarcinomas, mesothelioma, peritoneal cancer, fallopian tube cancer, cervical cancer, ovarian cancer, sarcomas, e.g. metastatic sarcoma, hematological neoplasms, thyroid cancer, salivary cancer, laryngeal (larynx) cancer, neuroblastoma, retinoblastoma, and testis (testicular) cancer.

Without wishing to be bound by theory, it is believed that antibodies of the present invention may be superior to prior art antibodies in terms of therapeutic efficacy, in particular in terms of speed of action and the concentration required (low or lower concentrations/doses of the antibodies of the invention have been shown to induce significant direct tumor cell killing compared to prior art antibodies and at rapid speed). In some embodiments, antibodies of the present invention have the ability to not induce the haemagglutination of red blood cells.

Whilst not wishing to be bound by theory, it is believed that the antibodies of the invention may function to stop progression of the cell cycle and hence tumour growth. Thus, in some embodiments antibodies (or binding proteins) of the invention are capable of causing the arrest of cell growth.

Efficacy of an antibody of the present invention in a relevant cancer model (a xenograft model of acute lymphoblastic leukaemia) has been demonstrated. In this model, a single administration of a concentration/dose of 1.33 nM of CO201-scFv resulted in a significant delay in tumour progression. The antibodies (and binding proteins) of the present invention thus represent an exciting development in the field of anti-CD47 therapeutic agents. Whilst not wishing to be bound by theory, it is believed that the antibodies of the invention may function to stop progression of the cell cycle and hence tumour growth. Thus, in some embodiments antibodies (or binding proteins) of the invention are capable of causing the arrest of cell growth. In some embodiments antibodies (or binding proteins) of the invention are capable of delaying the progression of cancer.

The administration of the binding proteins or antibodies in the therapeutic methods and uses of the invention is carried out in pharmaceutically, therapeutically, or physiologically effective amounts, to subjects (e.g. animals, e.g. human or non-human mammals) in need of treatment. Thus, said methods and uses may involve the additional step of identifying a subject in need of treatment. Appropriate and effective concentrations/doses to be administered can readily be determined by a person skilled in the art. Based on animal models used to date, exemplary concentrations/doses might be 0.05, 0.1, 1 or 5 nM to 10, 20, 30, 40 or 50 nM, e.g. at or about 0.05, 0.1, 1, 2, 4, 6, 10, 20, 30 or 40 nM. Treatment of diseases or conditions in accordance with the present invention (for example treatment of pre-existing disease) includes cure of said disease or condition, or any reduction or alleviation of disease, e.g. reduction in disease severity, or symptoms of disease.

The therapeutic methods and uses of the prevent invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis, or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

Suitable subjects for treatment in accordance with the present invention thus include any type of animal that can suffer from cancer, and more specifically cancers comprising tumour cells that express CD47.

Thus, the in vivo methods and uses as described herein are generally carried out in a mammal. Any mammal may be treated, for example humans and any livestock, domestic or laboratory animal. Specific examples include mice, rats, pigs, cats, dogs, sheep, rabbits, horses, cows and monkeys. Preferably, however, the mammal is a human.

Thus, the term “animal” or “patient” or “subject” as used herein includes any mammal, for example humans and any livestock, domestic or laboratory animal. Specific examples include mice, rats, pigs, cats, dogs, sheep, rabbits, horses, cows and monkeys. Preferably, however, the animal or patient or subject is a human. Thus, subjects or patients treated in accordance with the present invention will preferably be humans.

In another embodiment the subject is a subject having, or suspected of having (or developing), or potentially having (or developing) the disease or condition in question as described above.

Alternatively viewed, the present invention provides a method of treating or preventing cancer which method comprises administering to a patient in need thereof a therapeutically effective amount of an antibody (or binding protein) of the invention as defined herein. Embodiments of the therapeutic uses of the invention described herein apply, mutatis mutandis, to this aspect of the invention.

A therapeutically effective amount will be determined based on the clinical assessment and can be readily monitored. Preferred cancer therapies are as described elsewhere herein.

Further alternatively viewed, the present invention provides the use of an antibody (or binding protein) of the invention as defined herein in the manufacture of a medicament for use in therapy. Preferred therapy is the treatment or prevention of cancer as described elsewhere herein.

Embodiments of the therapeutic uses of the invention described herein apply, mutatis mutandis, to this aspect of the invention.

In some embodiments, the antibodies (or binding proteins) of the invention can be used in monotherapy. In other embodiments they can be used in combination with other standard cancer therapeutics.

The invention further includes kits comprising one or more of the antibodies, or compositions of the invention, or one or more of the nucleic acid molecules encoding the antibodies of the invention, or one or more expression vectors comprising the nucleic acid sequences of the invention, or one or more host cells or viruses comprising the expression vectors or nucleic acid sequences of the invention. Preferably said kits are for use in the methods and uses as described herein, e.g. the therapeutic, diagnostic or imaging methods as described herein. Preferably said kits comprise instructions for use of the kit components. Preferably said kits are for diagnosis or imaging, or treating or preventing diseases or conditions as described elsewhere herein, and optionally comprise instructions for use of the kit components to diagnose, image, treat or prevent such diseases or conditions. Equivalent embodiments with binding proteins of the invention are also provided.

The antibodies (or binding proteins) of the invention as defined herein may also be used as molecular tools for in vitro or in vivo applications and assays, for example binding assays or diagnostic assays. As the antibodies (and binding proteins) have one antigen binding site that binds to CD47, these can function as members of specific binding pairs and these molecules can be used in any assay where the particular CD47 binding pair member is required.

Thus, yet further aspects of the invention provide a reagent that comprises an antibody (or binding protein) of the invention as defined herein and the use of such antibodies (or binding proteins) as molecular tools, for example in in vitro or in vivo assays, for example for the detection of CD47, e.g. in a sample of interest.

The term “decrease” or “reduce” (or equivalent terms) as described herein includes any measurable decrease or reduction when compared with an appropriate control. Appropriate controls would readily be identified by a person skilled in the art and might include non-treated or placebo treated subjects or healthy subjects, or samples or assays where no antibody (or binding protein) of the invention is present, or where a control antibody (or binding protein), for example an isotype control antibody (or binding protein), is present. Preferably the decrease or reduction will be significant, for example clinically or statistically significant.

The term “increase” (or equivalent terms) as described herein includes any measurable increase or elevation when compared with an appropriate control. Appropriate controls would readily be identified by a person skilled in the art and might include non-treated or placebo treated subjects or healthy subjects, or samples or assays where no antibody (or binding protein) of the invention is present, or where a control antibody (or binding protein), for example an isotype control antibody (or binding protein), is present. Preferably the increase will be significant, for example clinically or statistically significant.

Preferably such increases (and indeed other increases, improvements or positive effects as mentioned elsewhere herein) or such decreases (and indeed other decreases, reductions or negative effects as mentioned elsewhere herein) are measurable increases, decreases, etc., (as appropriate), more preferably they are significant increases, decreases, etc., preferably clinically significant or statistically significant increases, for example with a probability value of ≤0.05 or <0.05, when compared to an appropriate control level or value (e.g. compared to an untreated or placebo treated subject or compared to a healthy or normal subject, or the same subject before treatment).

Methods of determining the statistical significance of differences between test groups of subjects or differences in levels of a particular parameter are well known and documented in the art. For example herein a decrease or increase in level of a particular parameter or a difference between test groups of subjects is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of ≤0.05 or <0.05.

Tables of Amino Acid Sequences Disclosed Herein and their Sequence Identifiers (SEQ ID NOs)

All amino acid sequences are recited herein from the N-terminus to the C-terminus in line with convention in this technical field.

TABLE A

| | | |
|---|---|---|
| SEQ ID | | |
| NO: | Description | Sequence |

CO-1 scFv

| | | |
|---|---|---|
| 1 | VH domain (nt) | CAGATCCAGTTGGTGCAGTCTGGACCTGAAC |
| | | TGAAGAAGCCTGGAGAGACAGTCAAGATCTC |
| | | CTGCAAGGCTTCTGGGTATACCTTCACAAACT |
| | | TTGGAATGCACTGGGTGAAGCAGGCTCCAGG |
| | | GAAGGGTTTAAAGTGGATGGGCTGGATAAAC |
| | | ACCTACACTGGAGAGCCAACATATACTGATGA |
| | | CTTCAAGGGACGGTTTGCCTTCTCTTTGGAGA |
| | | CCTCTGCCAGCACTGCCTATTTGCAGATCAAC |
| | | AGCCTCAAAAATGAGGACACGGCTACGTTTTT |
| | | CTGTACAAGAGGGGATTATAGGTACGGTGAC |
| | | TCCTGGGGCCAAGGCACCACTCTCACAGTCT |
| | | CCTCA |
| 2 | VL domain (nt) | GATGTTGTGATGACCCAAACTCCACTCTCCCT |
| | | GCCTGTCAGTCTTGGAGATCAAGCCTCCATCT |
| | | CTTGCAGATCTAGTCAGAGCCTTGTACACAGT |
| | | AATGGAAAAACCTACTTACATTGGTACCTGCA |
| | | GAAGCCAGGCCAGTCTCCAAAGCTCCTGATC |
| | | TACAGAGTTTCCAACCGATTTTCTGGGGTCCC |
| | | AGACAGGTTCAGTGGCAGTGGATCAGGGACA |
| | | GATTTCACACTCAAGATCAGCAGAATAGAGGC |
| | | TGAGGATCTGGGAGTTTATTTCTGCTCTCAAA |
| | | GTACACATGTTCCATTCACGTTCGGCTCGGG |
| | | GACAAAGTTGGAAATAAAA |
| 3 | VH domain (aa) | QIQLVQSGPELKKPGETVKISCKASGYTFTNFG |
| | | MHWVKQAPGKGLKWMGWINTYTGEPTYTDDF |
| | | KGRFAFSLETSASTAYLQINSLKNEDTATFFCTR |
| | | GDYRYGDSWGQGTTLTVSS |
| 4 | VL domain (aa) | DVVMTQTPLSLPVSLGDQASISCRSSQSLVHSN |
| | | GKTYLHWYLQKPGQSPKLLIYRVSNRFSGVPDR |
| | | FSGSGSGTDFTLKISRIEAEDLGVYFCSQSTHVP |
| | | FTFGSGTKLEIK |
| 5 | Heavy CDR1 | NFGMH |
| 6 | Heavy CDR2 | WINTYTGEPTYTDDFKG |
| 7 | Heavy CDR3 | GDYRYGDS |
| 8 | Light CDR1 | RSSQSLVHSNGKTYLH |
| 9 | Light CDR2 | RVSNRFS |
| 10 | Light CDR3 | SQSTHVPFT |
| 11 | Heavy FR1 | QIQLVQSGPELKKPGETVKISCKASGYTFT |
| 12 | Heavy FR2 | WVKQAPGKGLKWMG |
| 13 | Heavy FR3 | RFAFSLETSASTAYLQINSLKNEDTATFFCTR |
| 14 | Heavy FR4 | WGQGTTLTVSS |
| 15 | Light FR1 | DVVMTQTPLSLPVSLGDQASISC |
| 16 | Light FR2 | WYLQKPGQSPKLLIY |
| 17 | Light FR3 | GVPDRFSGSGSGTDFTLKISRIEAEDLGVYFC |
| 18 | Light FR4 | FGSGTKLEIK |
| 20 | linker | GGGGSGGGGSGGGGS |
| 21 | scFv (VH and VL | QIQLVQSGPELKKPGETVKISCKASGYTFTNFGM |
| | domains and linker) | HWVKQAPGKGLKWMGWINTYTGEPTYTDDFK |
| | | GRFAFSLETSASTAYLQINSLKNEDTATFFCTRGD |
| | | YRYGDSWGQGTTLTVSSGGGGSGGGGSGGGGS |
| | | DVVMTQTPLSLPVSLGDQASISCRSSQSLVHSNG |
| | | KTYLHWYLQKPGQSPKLLIYRVSNRFSGVPDRES |
| | | GSGSGTDFTLKISRIEAEDLGVYFCSQSTHVPFTF |
| | | GSGTKLEIK |

TABLE B
Humanized Antibodies

| | Heavy chain variable sequence | Light chain variable sequence |
|---|---|---|
| huCO-mAb | (SEQ ID NO:) | (SEQ ID NO:) |
| CO201 | 22 | 27 |
| CO202 | 23 | 27 |
| CO203 | 24 | 27 |
| CO204 | 25 | 27 |
| CO205 | 22 | 28 |
| CO206 | 23 | 28 |
| CO207 | 24 | 28 |
| CO208 | 25 | 28 |
| CO209 | 22 | 29 |
| CO210 | 23 | 29 |
| CO211 | 24 | 29 |
| CO212 | 25 | 29 |
| CO213 | 26 | 27 |

Humanized Variable Heavy domains (VH)
huCO-1 VH-v1
(SEQ ID NO: 22)
QVQLVQSGSELKKPGASVKVSCKASGYTFTNFGMHWVRQAPGQGLEWMGWINTYTGEP
TYTDDFKGRFVFSLDTSVSTAYLQISSLKAEDTAVYYCARGDYRYGDSWGQGTTVTVSS
huCO-1 VH-v2
(SEQ ID NO: 23)
QVQLVQSGSELKKPGASVKVSCKASGYTFTNFGMHWVRQAPGQGLEWMGWINTYTGEP
TYTDDFKGRFVFSLDTSVSTAYLQISSLKAEDTAVYYCTRGDYRYGDSWGQGTTVTVSS
huCO-1 VH-v3
(SEQ ID NO: 24)
QVQLVQSGSELKKPGASVKVSCKASGYTFTNFGMHWVRQAPGQGLKWMGWINTYTGEP
TYTDDFKGRFVFSLDTSVSTAYLQISSLKAEDTAVYYCTRGDYRYGDSWGQGTTVTVSS
huCO-1 VH-v4
(SEQ ID NO: 25)
QVQLVQSGSELKKPGASVKVSCKASGYTFTNFGMHWVRQAPGKGLKWMGWINTYTGEPT
YTDDFKGRFVFSLDTSVSTAYLQISSLKAEDTAVYYCTRGDYRYGDSWGQGTTVTVSS
huCO-1 VH-v5
(SEQ ID NO: 26)
QVQLVQSGSELKKPGASVKVSCKASGYTFTNFGMHWVRQAPGQGLKWMGWINTYTGEP
TYTDDFKGRFVFSLDTSVSTAYLQISSLKAEDTAVYYCARGDYRYGDSWGQGTTVTVSS
(CDRs shown underlined)
Humanized variable Light domains (VL)
huCO-1 VL-v1
(SEQ ID NO: 27)
DIVMTQTPLSLSVTPGQPASISCRSSQSLVHSNGKTYLHWYLQKPGQPPQLLIYRVSNRFS
GVPDRFSGSGSGTDFTLKISRVEAEDVGVYYCSQSTHVPFTFGQGTKLEIK
huCO-1 VL-v2
(SEQ ID NO: 28)
DIVMTQTPLSLSVTPGQPASISCRSSQSLVHSNGKTYLHWYLQKPGQSPQLLIYRVSNRFS
GVPDRFSGSGSGTDFTLKISRVEAEDVGVYYCSQSTHVPFTFGQGTKLEIK
huCO-1 VL-v3
(SEQ ID NO: 29)
DIVMTQTPLSLSVTPGQPASISCRSSQSLVHSNGKTYLHWYLQKPGQSPKLLIYRVSNRFS
GVPDRFSGSGSGTDFTLKISRVEAEDVGVYYCSQSTHVPFTFGQGTKLEIK
(CDRs shown underlined)
IgG4 CH2-CH3
(SEQ ID NO: 30)
PAPEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK
IgG4 - knob
(SEQ ID NO: 31)
SKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAK
GQPREPQVYTLPPSQEEMTKNQVSLWCLVKGFYPSDIAVEWESNGQPENNYKTTPPVLDS
DGSFFLYSRLTVDKSRWQEGNVFSCSVMHEALHNHYTQKSLSLSLGK
(knob mutation shown underlined)
IgG4 - hole
(SEQ ID NO: 32)
SKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAK
GQPREPQVYTLPPSQEEMTKNQVSLSCAVKGFYPSDIAVEWESNGQPENNYKTTPPVLDS
DGSFFLVSRLTVDKSRWQEGNVFSCSVMHEALHNHYTQKSLSLSLGK
(hole mutations shown underlined)
Hinge
(SEQ ID NO: 33)
SKYGPPCPSC
scFv-Fc(mono) CO201 Fc knob
(SEQ ID NO: 34)
QVQLVQSGSELKKPGASVKVSCKASGYTFTNFGMHWVRQAPGQGLEWMGWINTYTGEP
TYTDDFKGRFVFSLDTSVSTAYLQISSLKAEDTAVYYCARGDYRYGDSWGQGTTVTVSSG
GGGSGGGGSGGGGSDIVMTQTPLSLSVTPGQPASISCRSSQSLVHSNGKTYLHWYLQKP
GQPPQLLIYRVSNRFSGVPDRFSGSGSGTDFTLKISRVEAEDVGVYYCSQSTHVPFTFGQG
TKLEIKSKYGPPCPSCPAPEFLGGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSIEKTI
SKAKGQPREPQVYTLPPSQEEMTKNQVSLWCLVKGFYPSDIAVEWESNGQPENNYKTTPP
VLDSDGSFFLYSRLTVDKSRWQEGNVFSCSVMHEALHNHYTQKSLSLSLGK
(knob mutation shown underlined)
scFv-Fc(mono) CO201 Fc hole
(SEQ ID NO: 35)
SKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAK
GQPREPQVYTLPPSQEEMTKNQVSLSCAVKGFYPSDIAVEWESNGQPENNYKTTPPVLDS
DGSFFLVSRLTVDKSRWQEGNVFSCSVMHEALHNHYTQKSLSLSLGK
(hole mutations shown underlined)
Alternative hinge
(SEQ ID NO: 36)
SKYGPPCPPC

The invention will now be further described in the following non-limiting Examples with reference to the following drawings:

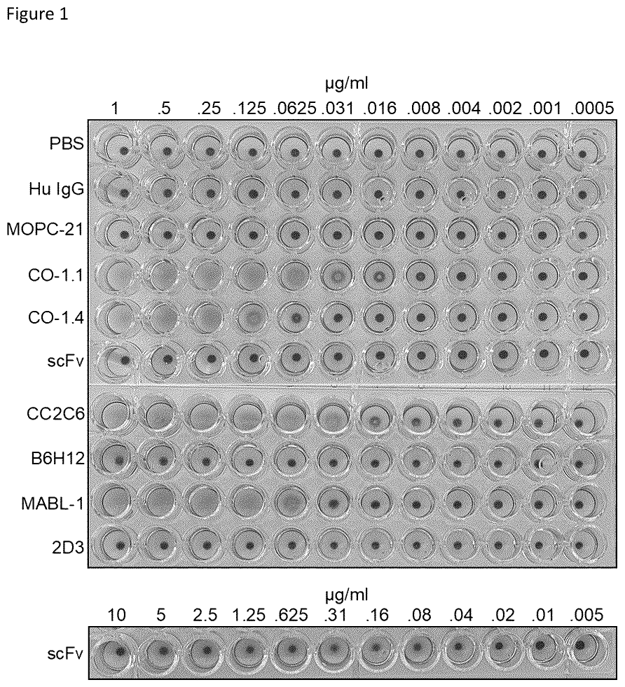

FIG. 1: CO-1 scFv induces no hemagglutination, even at high concentrations. (Upper panel) RBCs (2% v/v in PBS) were incubated with CO-1 scFv (scFv), anti-CD47 (CO-1.1, CO-1.4, CC2C6, B6H12, MABL-1, or 2D3) or isotype controls (human IgG or MOPC-21) as indicated at concentrations ranging from 0.0005-1 μg/ml. (Lower panel) RBCs (2% v/v in PBS) were incubated with CO-1 scFv (scFv) at concentrations ranging from 0.005-10 μg/ml. (Upper and lower panel) A small punctate circle indicates no hemagglutination, while a diffuse hazy pattern indicates hemagglutination.

FIG. 2: Binding of CO-1 scFv to Jurkat cells. Jurkat cells (2×10⁵ cells) were incubated with increasing concentrations (range: 0.1 ng/ml-100 μg/ml) of either CO-1 scFv, or human IgG isotype control followed by incubation with 1 μg/ml His₆-FITC. Binding was determined by measuring % FITC-positive cells. Each data point represents the MFI±S.D. of two replicates. EC₅₀ of CO-1 scFv=0.05 ng/ml.

FIG. 3: CO-1 scFv induces potent PCD in cell lines derived from hematological malignancies. Jurkat (A), MOLT-4 (B), or CCRF-CEM (C) cells were treated with CO-1, CO-1 scFv, or human IgG isotype control for the indicated time points and concentrations, followed by staining with Annexin V and 7-AAD. The data represents the sum of cells undergoing early (Annexin V+7-AAD−) and late (Annexin V+7-AAD+) PCD. (A and C) n=1, (B) The data represent the mean±S.D. of two independent experiments.

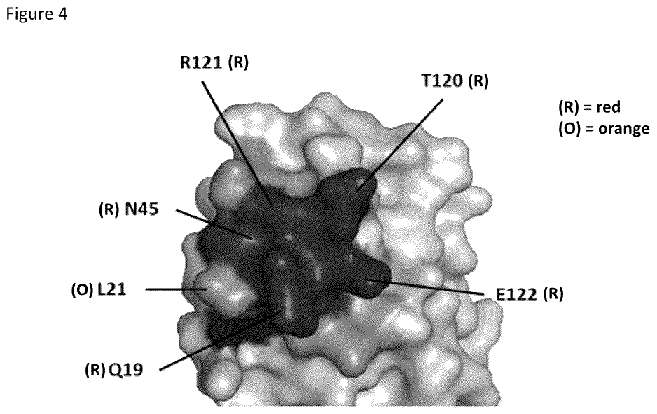

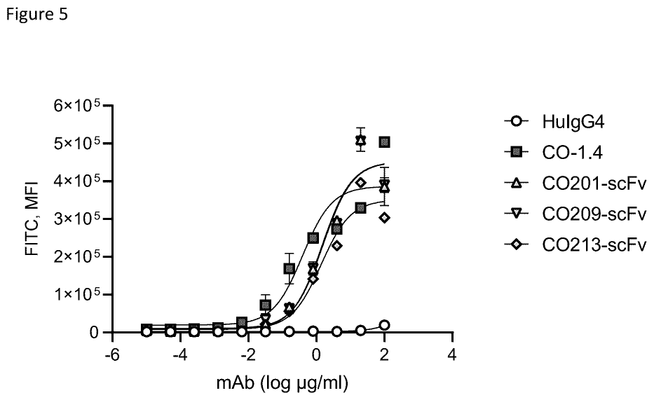

FIG. 4: Epitope surface map of the CD47 residues bound by CO-1 antibody. Residues Q19, N45, T120, R121, and E122 are labelled on the CD47 protein crystal structure as some of the specific residues (labelled red (R)) that form the binding epitope of the CO-1 antibody.

FIG. 5: Binding of huCO-scFv to Jurkat cells. Jurkat cells incubated with increasing concentrations of FITC-conjugated huCO-scFv. FITC-intensity was analyzed by flow cytometry.

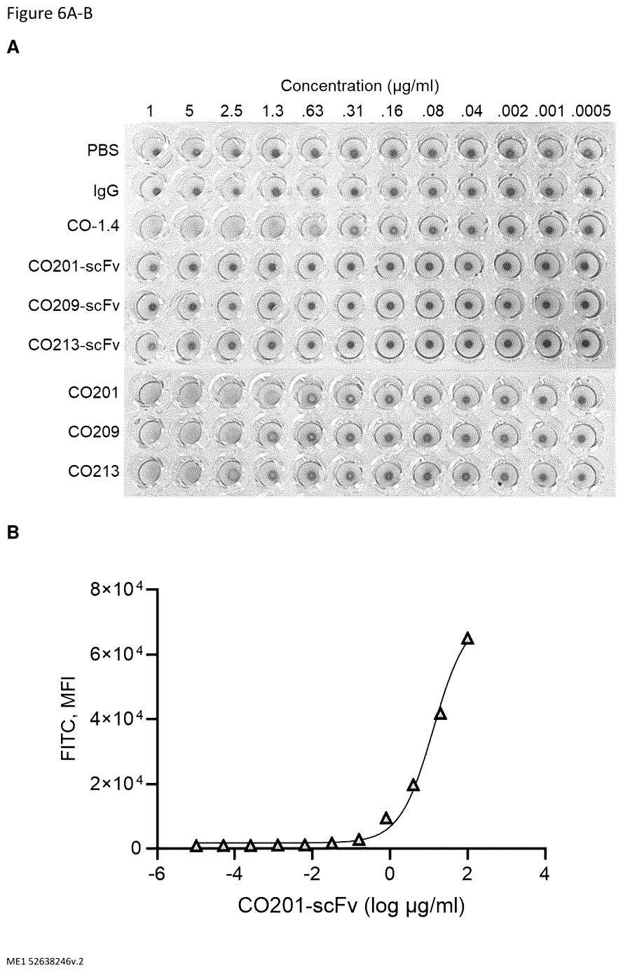

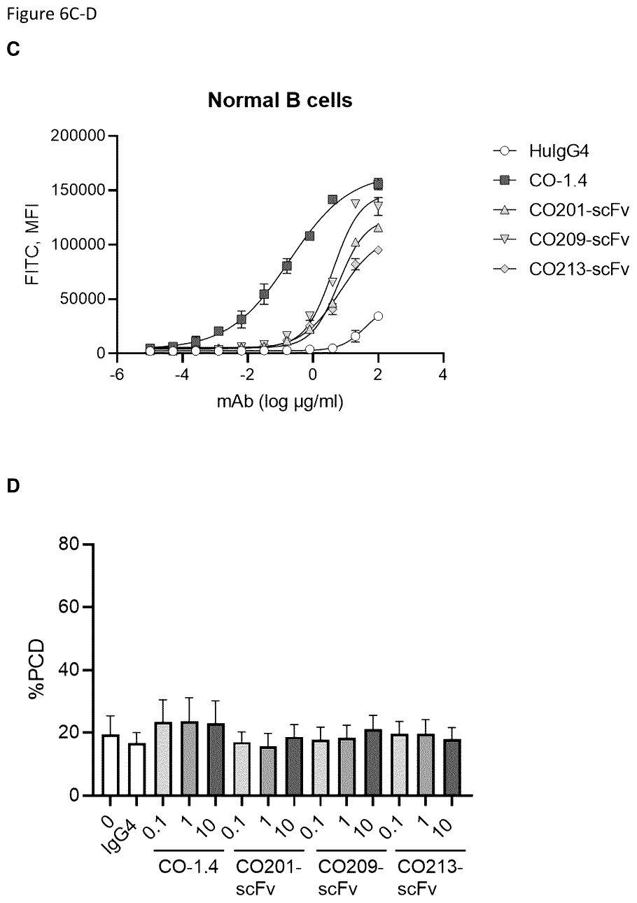

FIG. 6: Hemagglutination and binding of red blood cells (RBCs) by huCO-scFv. (A) 2% RBCs (v/v in PBS) were incubated with increasing concentrations of the indicated antibodies or fragments for 30 minutes. A small punctate circle indicates no hemagglutination, while a diffuse hazy pattern indicates hemagglutination. (B) 2% RBCs (v/v in PBS) were incubated with increasing concentrations of FITC-conjugated CO201-scFv and analyzed by flow cytometry. (C and D) B cells were isolated from buffycoats of healthy human donors using CD19 Dynabeads and stimulated with 1 μg/ml CpG and RP105 for 24 hours. (C) B cells were incubated with the indicated antibodies or fragments at increasing concentrations and analyzed for binding by flow cytometry. (D) B cells were incubated with the indicated antibodies or fragments at concentrations in μg/ml for 3 hours, followed by staining with Annexin V and 7-AAD. % PCD=Annexin V-positive cells.

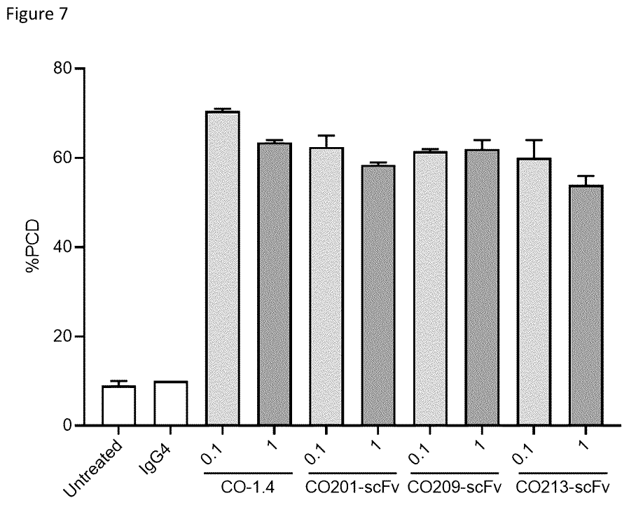

FIG. 7: Induction of PCD by CO scFv fragments. Jurkat cells were treated with 0.1 or 1 μg/ml of the indicated fragments or CO-1 for 3 hours followed by Annexin V and 7-AAD staining. % PCD=Annexin V-positive cells. Data are presented as mean±SEM, n=2.

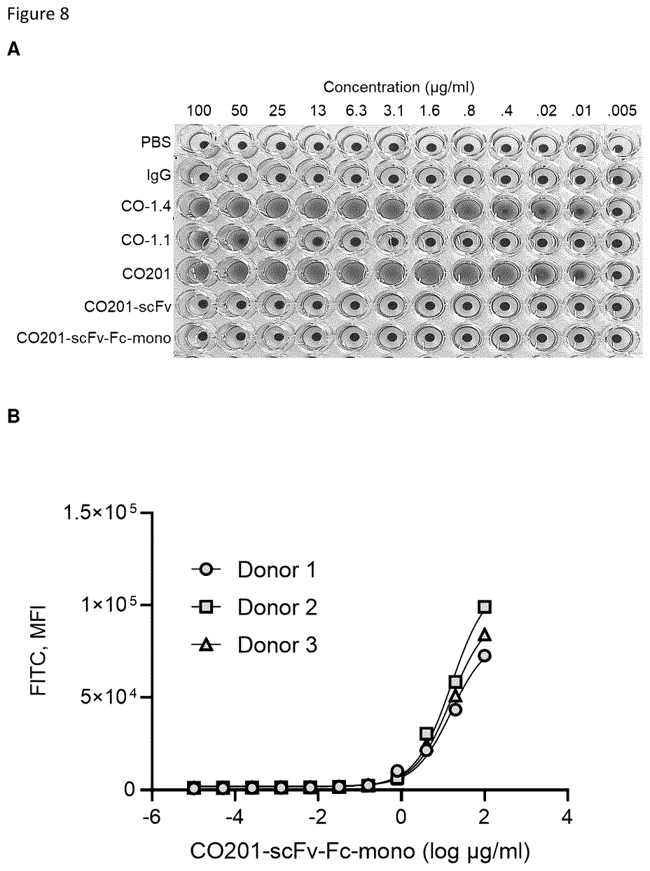

FIG. 8: Hemagglutination and binding of red blood cells (RBCs) by huCO201-scFv-Fc-mono. (A) 2% RBCs (v/v in PBS) were incubated with increasing concentrations of the indicated antibodies or fragments for 30 minutes. A small punctate circle indicates no hemagglutination, while a diffuse hazy pattern indicates hemagglutination. (B) 2% RBCs (v/V in PBS) from three donors were incubated with increasing concentrations of FITC-conjugated CO201-scFv-mono and analyzed by flow cytometry.

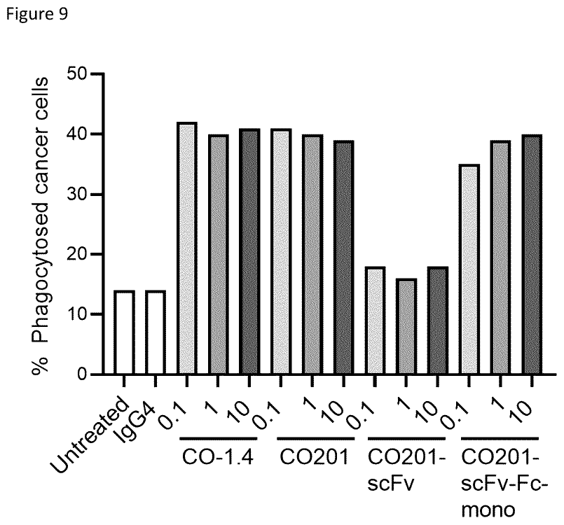

FIG. 9: Induction of phagocytosis by huCO-scFv-Fc-mono. Jurkat cells (5×10⁵ cells/ml) were co-cultured with RAW264, and treated with the indicated antibodies for 2 hours. 1% Phagocytosed cancer cells=% Jurkat-CFSE⁺RAW-DiO⁺ cells. Data are from one experiment.

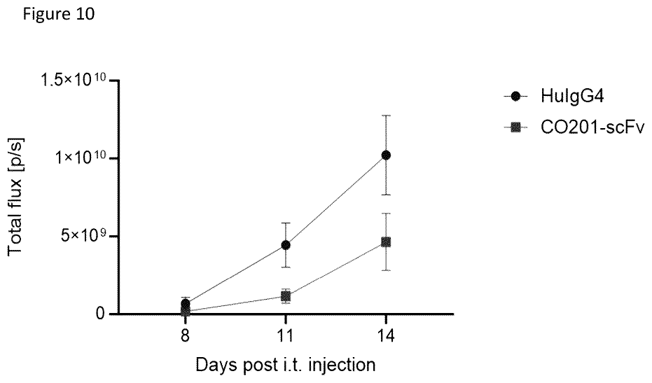

FIG. 10: CO201-scFv delays the progression of BCP-ALL in vivo. 6-8 weeks old NSG mice were injected IT with lentivirally transduced Reh cells. The cancer progression was followed by noninvasive in vivo imaging of the luminescence signal emitted by the Reh cells as described in Materials and Methods. Establishment of the xenograft was confirmed on day 8 after IT injection, followed by injection of 1.33 nM of CO201-scFv or human IgG4 isotype control. The effect of the treatment was determined on day 11 after IT injection and the cancer progression was measured again on day 14. Xenograft luciferase activity [photons per second, (p/s)] of each treatment was measured at the indicated time points. Each dot represents the mean luminescence signal (p/s) from the mice in the indicated treatment group, while vertical bars indicate the standard error of the mean of the 5 xenograft mice in each treatment group.

### EXAMPLES

### Example 1: Functional Characteristics of Monovalent Anti-CD47 Antibodies

### Materials and Methods

### Reagents and Antibodies

Reagents and antibodies used in the study are listed in Table 1 and 2, respectively.

The nucleotide and amino acid sequences of the heavy and light variable domains of one preferred CD47 antibody of the invention is shown in Table A. The antibody is an scFv monomeric fragment designated as CO-1 scFv. It has been derived from a murine full length IgG1 kappa antibody called CO-1 (mCO-1) which is obtained from a hybridoma. The CDR and framework regions of the light and heavy chains of the scFv fragment of mCO-1 are shown in Table A.

### Cell Lines and Culture Conditions

The human cancer cell lines used in this project were purchased from the American Type Culture Collection (ATCC). Jurkat (clone E6-1), MOLT-4, and CCRF-CEM were cultured in RPMI1640 medium (Lonza cat.no. BE-12-702F/12).

All cell culture media were supplemented with 10% (v/v) fetal bovine serum (FBS, BioNordika cat.no. FB-1001/500), and 1% (v/v) penicillin/streptomycin (P/S, ThermoFisher cat.no. 15140122), and all cell lines were maintained at 37° C. in a humidified atmosphere with 95% air and 5% CO2.

### Red Blood Cell (RBC) Isolation

Human whole blood was collected into heparin-coated tubes (approximately 5 ml), and diluted in RBC wash buffer (0.05% BSA, 1 mM EDTA in PBS) to a total volume of 50 ml. RBCs were isolated from human whole blood by centrifugation 1800×g for 10 minutes, then resuspended in wash buffer to a total volume of 50 ml. Wash was repeated 3 times, and after the last wash the RBC pellet was resuspended in PBS to create a 2% (v/v) RBC solution.

### Hemagglutination Assay

Increasing concentrations of up to 10 μg/ml) of CD47 antibodies (or controls) were added to a round-bottom 96-well plate. Following that, a solution of 2% (v/v) freshly isolated RBCs were added to each well and incubated in a standard cell incubator for 30-60 minutes or until cells have settled in the bottom of the well. A diffuse hazy pattern indicates hemagglutination, while a small punctate circle indicates no hemagglutination.

### Flow Cytometry

All flow cytometry analyses were performed on a NovoCyte (Agilent Technologies Inc.) equipped with 3 lasers (405, 488, 605 nm) and 13 detection channels. Data were analyzed using the NovoExpress software (Agilent Technologies Inc.).

### Antibody Binding Assays

PBS-washed cancer cell lines or RBCs were pelleted and washed with washing buffer (3% (w/v) BSA, 1% (w/v) sodium azide in PBS) and incubated with Human BD Fc Block for 10 minutes before being plated into a round-bottom 96 well plate (2.5×10⁵ cells per well). The cells were then incubated with various concentrations of CO-1 scFv, or appropriate isotype controls as indicated for 1 h on ice with slight agitation. All samples were prepared in duplicates. Cells were washed twice, resuspended in cold His staining solution (1 μg/ml His₆-FITC in washing buffer), and incubated for 30 minutes on ice with slight agitation. Cells were analyzed by flow cytometry after two washes. Excitation wavelength FITC: 488 nm, detection: 530/30 nm.

### Annexin V and 7-AAD Staining

For programmed cell death (PCD) analyses, cells were diluted to 5×10⁵ cells per ml in supplemented media and seeded into 24 well plates, 1 ml per well. They were then incubated with various concentrations of CO-1 scFv fragments or appropriate isotype controls for different durations (30 minutes, 1 hr, 2 hrs, 3 hrs) at standard cell culture conditions. Following the incubation, cells were harvested and stained with Annexin V eFlour™ 450 and 7-AAD according to manufacturers' protocol. Cells were analyzed immediately by flow cytometry. Annexin V⁺7-AAD were considered early apoptotic (in the early stages of PCD), while late apoptotic cells (cells in the late stages of PCD) were Annexin V⁺ and 7-AAD⁺. Excitation Annexin V 405 nm, detection 445/45 nm, while 7-AAD was excited at 488 nm and detected at 675/30 nm.

### Statistical Analyses

Graphs are presented as mean values from independent experiments as specified in the figure legends. Error bars indicates the standard deviation (S.D.).

Statistical analyses were performed by doing the paired two-tailed Student t test using the GraphPad Prism 9 software (Graph-Pad Software Inc.). Groups were found to be significantly different from each other when the P value was below 0.05.

Curve fitting was done using four parameter nonlinear regression in GraphPad Prism.

TABLE 1
List of reagents

| Name/ID | Supplier | Cat. no. |
|---|---|---|
| Annexin V Apoptosis Detection Kit eFluor ™ 450 | ThermoFisher | 88-8006-74 |
| Bovine serum albumin (BSA) | Merck | A7030 |
| Ethylene Diamine Tetra Acetic Acid solution (EDTA) | Merck | 03690 |
| FITC conjugation kit | Abcam | ab188285 |
| Human BD Fc Block | BD Biosciences | 564219 |
| Sodium azide | Merck | S2002 |

TABLE 2
List of antibodies

| Name/ID | Target | Isotype | Species | Supplier | Cat. no. |
|---|---|---|---|---|---|
| 2D3 | CD47 | IgG1 κ | Mouse | ThermoFisher | 14-0478-82 |
| B6H12 | CD47 | IgG1 κ | Mouse | ThermoFisher | 14-0479-82 |
| CC2C6 | CD47 | IgG1 κ | Mouse | Santa Cruz | sc-21786 |
| CO-1.1 | CD47 | IgG1 | Chimeric | Comparator Antibody | N/A |
| CO-1.4 | CD47 | IgG4 | Chimeric | Comparator Antibody | N/A |
| His₆, FITC | 6X His tag | IgG | Rabbit | Abcam | ab1206 |
| Human IgG | IgG | IgG | Human | ThermoFisher | 27102 |
| MABL-1 | CD47 | IgG1 κ | Mouse | Creative Biolabs | HPAB-1278WJ |
| mCO-1 | CD47 | IgG1 κ | Mouse | Comparator Antibody | N/A |
| MOPC-21 | IgG1 | IgG1 κ | Mouse | ThermoFisher | MA110407 |
| MOPC-21, FITC | IgG1 | IgG1 κ | Mouse | ThermoFisher | MA110413 |

### Results

### Assessment of RBC Hemagglutination by CO Fragments

Since anti-CD47 antibodies such as CC2C6 can induce hemagglutination in RBCs, we assessed the ability of the CO-1 scFv fragment and several other CD47 antibodies to induce hemagglutination in RBCs derived from healthy human donors. A diffuse hazy pattern indicates hemagglutination, while a small punctate circle indicates no hemagglutination, and as seen in FIG. 1, the CO-1 scFv fragment induced no hemagglutination even at concentrations as high as 10 μg/ml, while the comparator full-length divalent CO antibodies (CO-1.1, a full length IgG1 format of the CO-1 antibody, and CO-1.4, a full length IgG4 format of the CO-1 antibody), and other CD47 antibodies induced hemagglutination.

Binding of CO-1 scFv to Jurkat Cells

Having established that CO-1 scFv does not induce RBC hemagglutination, we proceeded with elucidating the binding of CO-1 scFv in the human T cell leukemia cell line Jurkat. The cells were incubated with increasing concentrations of CO-1 scFv, or isotype control, followed by incubation with an anti-His, FITC secondary antibody. As seen in FIG. 2, CO-1 scFv binds to Jurkat cells with high affinity. The EC₅₀ for CO-1 scFv was analyzed by four parameter linear curve fitting in GraphPad and determined to be 0.05 ng/ml.

Induction of PCD by CO-1 scFv

Several anti-CD47 antibodies have been shown to induce PCD in a wide range of tumor cells, and we thus tested the ability of the CO-1 scFv fragment to induce PCD by staining cancer cells with Annexin V and 7-AAD after incubation with the fragment for different durations and at different concentrations. Cells that stained positive for Annexin V and negative for 7-AAD (Annexin V⁺ 7-AAD⁻) were considered early apoptotic, while cells staining positive for both dyes (Annexin V⁺ 7-AAD⁺) were considered late apoptotic. Summing both early and late apoptotic cells, provided the total percentage of the cell population that was undergoing PCD. Treatment of Jurkat cells with CO-1 scFv causes a strong induction of PCD as early as after 30 minutes of treatment with the fragment (FIG. 3A) and also after 3 hours (FIG. 3A). PCD of MOLT-4 cells (FIG. 3B) and CCRF-CEM cells (FIG. 3C) was also observed. The effect of a full length divalent CO-1 antibody is shown for comparison. The ability of the scFv fragment of CO-1 to be able to induce PCD is surprising and advantageous, as there is suggestion in the art that a divalent interaction might be required for PCD. This property is particularly advantageous in combination with the lack of HA observed as shown above.

### Example 2: SPR Analysis of Antibodies Against Recombinant CD47 to Assess Binding Affinity

The affinity of CO-1 scFv to CD47 was determined via surface plasmon resonance (SPR) on a Biacore S200 system. Recombinant biotinylated CD47 was immobilized on an SA streptavidin chip to a level of 100 RU (in 10 mM NaAc pH 5.0). CO-1 scFv reacted with recombinant CD47 at gradient concentrations

### Analysis 1

### Instrument: Biacore S200

### Material:

SA chip (Streptavidine)
recCD47: SinoBiological (12283-H27H-B); 17 kDa, C-term His- and AVI-tag, biotinylated
Antibody Fragments: scFv (0.11 mg/ml)
All proteins stored at −80° C.
Buffer: 20 mM Phosphate buffer pH 7.4/2.7 mM KCl/137 mM NaCl/0.05% P20 Single-cycle Procedure:
recCD47 immobilized to a level of 100 RU (in 10 mM NaAc pH 5.0)
Regeneration condition: 60 sec 10 mM glycin pH 1.5
Analyte (antibody fragments) run in single-cycle mode
Injection order low to high concentration: 10 nM/5 nM/2.5 nM/1.25 nM/0.625 nM/0.3125 nM/0.1562 nM/0.07813 nM/0.039 nM
Flow 30 μl/min, 120s on, 180s off-1800s off after last injection
Temperature: 25° C.

### Results

A 1:1 binding model was used to fit the data. All curves are well fitted to the 1:1 model. Table 3 shows the values obtained for the binding on (column 2) and off (column 3) rates ka and kd, respectively. The affinity constant KD is calculated as kd/ka (columns 4 and 5), 180 pM. Thus, the scFv fragment shows a high binding affinity to CD47.

TABLE 3

| Analyte | kₐ (1/Ms) | k_{d} (1/s) | K_{D} (M) | |
|---|---|---|---|---|
| scFv | 3.1 × 10⁷ | 5.6 × 10⁻³ | 1.79 × 10⁻¹⁰ | 180 pM |

### Example 3: Epitope Mapping of CO-1

Epitope mapping of an IgG form of the CO-1 antibody on human CD47 was carried out by Deeptope SAS, France, using DMS (Deep Mutational Scanning), see for example as described in Sierocki et al., 2021, PLOS Negl Trop Dis., 15 (3): e0009231. See also Van Blarcom et al., 2015, JMB, 427:6(B):1513-1534 and Medina-Cucurella and Whitehead, 2018, Methods Mol. Biol., 1764:101-121.

### Principle of Epitope Mapping Using DMS (Deep Mutational Scanning)

DMS is a mutagenesis method that aims to perform all possible mono-substitutions on all selected residues within a given protein sequence. The DMS library is obtained in the form of DNA coding for the protein under study. In this library, each DNA strand contains a codon that is mutated with respect to the parental sequence.

This DMS DNA library is integrated into an expression plasmid specifically designed to express recombinant proteins on the yeast surface. Yeasts are then transformed and induced to allow the expression of the mono-mutated proteins on their surface. This new library (called display library) is screened by flow cytometry using fluorescent reporters to reveal the expression of the protein (anti-tag fluorescent antibody) as well as the binding of the protein to its partner (fluorescent partner).

For epitope mapping, the ideal case is to have two antibodies with compatible epitopes that can bind together on the same antigen. In this way, each of the two antibodies acts as a conformational control of the mutated antigen for the other antibody. Indeed, mono-substitutions made on the antigen can have 4 types of effects:
1. Loss of affinity for the first antibody, while retaining binding for the second: this is a mutation made within the epitope of the first antibody.
2. Loss of affinity for the second antibody, while retaining binding for the first: this is a mutation in the epitope of the second antibody.
3. Loss of affinity for both antibodies: this is a so-called “destructuring” mutation which affects the conformation of the antigen and thus prevents the binding of both antibodies.
4. No effect: the mutation is not present in the epitope of one of the two antibodies and does not cause a significant change in the conformation of the antigen.

Following flow cytometry analysis, the yeast population that has lost affinity for the antibody of interest while retaining binding for the second antibody is sorted. The plasmids contained in this yeast population are extracted and sequenced by high-throughput sequencing. Analysis of the sequencing data allows the identification of mutations that have affected the binding of the antibody to its target. Thus, this analysis allows to identify the important positions on the antigen for the binding of the antibody of interest: i.e. its epitope.

### Materials and Methods

The antigen used for the DMS analysis was human CD47 (123 amino acids expressed from the glutamine 19 to the Glutamic acid 141), see SEQ ID NO:19. Mutated versions of this antigen are expressed on yeast in the form of DMS DNA libraries. The first antibody used was CO-1. The second antibody was 2D3 (ThermoFisher, Cat no-14-0478-82), which is another anti-CD47 antibody that has been shown not to compete with CO-1 for binding to CD47. Thus, these are an appropriate pair of antibodies to use in the DMS analysis.

DMS was performed on 2 areas of CD47: Library 1 [amino acid 19 to 80] and Library 2 [amino acid 81 to 141]. These 2 libraries were transformed into yeast. Unsorted yeasts from the 2 generated libraries were sequenced to verify the efficiency of mutagenesis. 100% of the expected single mutants were sequenced for all libraries. The 2 DMS libraries were successfully generated and cloned into yeasts. Each library contains approximately 1200 single amino acid mutants and 2000 DNA codon mutants. Members of the libraries encode each of the 20 appropriate amino acids at each mutated position.

### Results

A DMS map is generated, see Table 4. Every position which has an impact on the binding of the CO-1 antibody upon mutation are classified in three categories:
High impact: between 19 and 14 mutations are forbidden (Red/R). Positions classified in this category are considered likely to be in direct interaction with the IgG—here CO-1.
Medium impact: between 13 and 7 mutations are forbidden (orange/O)
Low impact: between 2 and 6 mutations are forbidden (yellow/Y).

An analysis of the structure of CD47 (AF-Q08722-F1, Alphafold) is performed in order to decipher between “structural residues” that are buried inside the structure and the epitope residues that are exposed to solvent. Residues coloured in grey in Table 4 are classified as “structural residues” and are not believed to be part of the epitope. Their mutation induces a soft structural change that is enough to lose the binding for the antibody of interest but not enough to lose the binding of the second, so called “conformational control” antibody (in this case, the 2D3).

The residues marked*(corresponding to high impact—R residues, but not structural residues) are believed to form the CO-1 epitope. The epitope is also depicted in FIG. 4.

TABLE 4
Classification of the positions showing a significant impact on the binding of the
CO-1 antibody when mutated.

| | | | | | | | | | | | | | | | | | | | | | | | |
|---|---|---|---|---|---|---|---|---|---|---|---|---|---|---|---|---|---|---|---|---|---|---|---|
| 19 | 20 | 21 | 22 | 30 | 43 | 45 | 46 | 47 | 54 | 55 | 57 | 69 | 70 | 72 | 73 | 74 | 76 | 77 | 78 | 79 | 81 | 82 | 84 |
| Q | [symbol] | L | [symbol] | [symbol] | [symbol] | N | M | E | [symbol] | Y | K | D | [symbol] | L | N | R | T | V | P | T | [symbol] | S | [symbol] |
| 18 | 16 | 13 | 12 | 2 | 3 | 19 | 0 | 7 | 0 | 0 | 0 | 0 | 0 | 0 | 0 | 0 | 0 | 0 | 0 | 0 | 0 | 0 | 0 |
| R | R | O | O | Y | Y | R | W | O | W | W | W | W | W | W | W | W | W | W | W | W | W | W | W |
| * | | | | | | * | | | | | | | | | | | | | | | | | |
| 115 | 116 | 117 | 118 | 119 | 120 | 121 | 122 | 123 | 124 | 127 | 129 | | | | | | | | | | | | |
| E | [symbol] | T | E | L | T | R | E | G | E | [symbol] | [symbol] | | | | | | | | | | | | |
| 13 | 2 | 4 | 10 | 0 | 15 | 18 | 19 | 15 | 2 | 9 | 11 | | | | | | | | | | | | |
| 0 | Y | Y | 0 | W | R | R | R | R | Y | O | O | | | | | | | | | | | | |
| | | | | | * | * | * | * | | | | | | | | | | | | | | | |

Top line: the numbering of the amino acid residues in the CD47 molecule of SEQ ID NO: 19.
Second line: amino acid residues; Grey shading = structural residues.
Third line: the number of mutations that are forbidden at that position, between 2 and 19.
Fourth line: R = red, high impact, between 19 and 14 mutations are forbidden; O = orange, medium impact, between 13 and 7 mutations are forbidden; Y = yellow, low impact, between 2 and 6 mutations are forbidden; W = white, no impact.
Bottom line: * denotes the residues believed to be part of the CO-1 epitope.

### Example 4: Functional Characterization of Monovalent Humanized Anti-CD47 scFv Fragments

### Materials and Methods

### Reagents and Antibodies

Reagents and antibodies used in the study are listed in Table 5 and 2, respectively.

The nucleotide and amino acid sequences of the heavy and light variable domains of one preferred CD47 antibody of the invention is shown in Table A. The antibodies being characterized in this Example are exemplary humanized scFv fragments targeting CD47 and are designated as CO201-scFv, CO209-scFv, CO213-scFv (hereafter collectively referred to as huCO-scFv). All fragments have been constructed using the CDRs from a murine full length IgG1 kappa antibody called CO-1 (mCO-1) which is obtained from a hybridoma (the CDR and framework regions of this murine antibody are shown in Table A). The CDR and framework regions of the light and heavy chains of the humanized fragments are shown in SEQ ID NO:s 22 to 26 (heavy chains) and 27-29 (light chains) and Table B. The CO201-scFv has also been engineered with an Fc (an IgG4 Fc) and is designated CO201-scFv-Fc-mono. This fragment has an Fc region but only one scFv fragment (i.e. monovalent) which is attached to one of the chains of the Fc. This fragment is conveniently engineered using “knobs into hole” mutations in order to encourage the correct association of the two chains. The sequence of one of the chains of this construct (the “knob” chain) is provided as SEQ ID NO:34 and comprises the CO201 scFv fragment-hinge-CH2-CH3, together with an appropriate “knob” mutation. The sequence of the other chain of this construct (the “hole” chain) is provided as SEQ ID NO:35 and comprises a hinge-CH2-CH3 fragment, together with an appropriate “hole” mutations. When these two heavy chain constructs are expressed the chains will dimerise and the complexes formed of knob-hole dimers are selected which comprise a monovalent scFv-Fc construct, i.e. an Fc region with a single (monovalent) scFv (i.e. a monovalent scFv-Fc fusion). The human IgG4 isotype control is obtained from SinoBiological (cat no: HG4K).

### Cell Lines and Culture Conditions

Jurkat (clone E6-1) cells were purchased from the American Type Culture Collection (ATCC) and were cultured in RPMI1640 medium supplemented with 10% (v/v) fetal bovine serum (FBS), and 1% (v/v) penicillin/streptomycin (PS). The cells were maintained at a density between 0.4 and 1.6×10⁶ cells per mL at 37° C. in a humidified atmosphere with 95% air and 5% CO₂.

### Red Blood Cell (RBC) Isolation

As for Example 1.

### Isolation of Normal B Cells

B cells were isolated from buffy coats of healthy human donors using CD19 Dynabeads and DETACHaBEADs according to manufacturers' instruction. Following isolation, the B cells were seeded into 24 well plates and stimulated with 1 μg/ml CpG and RP105 for 24 hours before downstream experiments.

### Hemagglutination Assay

Increasing concentrations of up to (100 μg/ml) of huCO-scFv (or controls) were added to a round-bottom 96-well plate. Following that, a solution of 2% (v/v) freshly isolated RBCs were added to each well and incubated in a standard cell incubator for 30-60 minutes or until cells have settled in the bottom of the well. A diffuse hazy pattern indicates hemagglutination, while a small punctate circle indicates no hemagglutination.

### Flow Cytometry

As for Example 1.

### Antibody Binding Assays

DPBS-washed cancer cell lines or RBCs were pelleted and washed with washing buffer (3% (w/v) BSA, 1% (w/v) sodium azide in DPBS) and incubated with Human BD Fc Block for 10 minutes before being plated into a round-bottom 96 well plate (2.5×10⁵ cells per well). The cells were then incubated with various concentrations of FITC-conjugated huCO-scFv, or human IgG4 isotype control as indicated for 1h on ice with slight agitation. All samples were prepared in duplicates. The cells were analyzed by flow cytometry after three washes in washing buffer. Excitation wavelength FITC: 488 nm, detection: 530/30 nm.

### Annexin V and 7-AAD Staining

As for Example 1 but incubating with huCO-scFv or controls for 3 hours.

### Statistical Analyses

Graphs are presented as mean values from independent experiments as specified in the figure legends. Error bars indicate the standard error of the mean (SEM). Statistical analyses were performed by doing the paired two-tailed Student t test using the GraphPad Prism 9 software (Graph-Pad Software Inc.). Groups were found to be significantly different from each other when the Pvalue was below 0.05. Curve fitting was done using four parameter nonlinear regression in GraphPad Prism.

TABLE 5
List of reagents

| Name/ID | Supplier | Cat. no. |
|---|---|---|
| Annexin V Apoptosis Detection Kit eFluor ™ 450 | ThermoFisher | 88-8006-74 |
| Bovine serum albumin (BSA) | Merck | A7030 |
| CellTrace ™ Violet Cell Proliferation Dye | ThermoFisher | C34557 |
| DETACHaBEAD ™ CD19 Kit | ThermoFisher | 12506D |
| Dulbecco's Modified Eagle Medium (DMEM): F-12 with | EuroClone | ECM0095L-12 |
| L-Glutamine and 25 mM Hepes | | |
| Dulbecco's phosphate-buffered saline (DPBS) | ThermoFisher | 14190250 |
| Dynabeads ® CD19 pan B | ThermoFisher | 11143D |
| Ethylene Diamine Tetra Acetic Acid solution (EDTA) | Merck | 3690 |
| Fetal bovine serum (FBS) | ThermoFisher | A3160802 |
| Lightning Link ® FITC conjugation kit | Abcam | ab188285 |
| Human BD Fc Block | BD Biosciences | 564219 |
| RPMI1640 | EuroClone | ECB2000L |
| Penicillin-Streptomycin | Biosera | LM-A4118 |
| Sodium azide | Merck | S2002 |
| Vybrant ™ DiO Cell-Labelling Solution | ThermoFisher | V22886 |

### Results

Binding of huCO-scFv to Cancer Cells and Human RBCs

Binding of huCO-scFv to Jurkat cells was determined by flow cytometry compared to a human IgG4 isotype control. The huCO-scFv bound specifically and with high affinity to Jurkat cells (FIG. 5).

Since anti-CD47 antibodies such as CC2C6 can induce hemagglutination in RBCs, we assessed the ability of the huCO-scFv and other CO-antibodies to induce hemagglutination in RBCs derived from healthy human donors. A diffuse hazy pattern indicates hemagglutination, while a small punctate circle indicates no hemagglutination, and as seen in FIG. 6A, the humanized fragments induced no hemagglutination up to 1 μg/ml as opposed to the full humanized (CO201, CO209, CO213) and chimeric (CO-1.4) IgG4 molecules. A flow-based binding assay also confirmed this low binding affinity of CO201-scFv (FIG. 6B). We also assessed the ability of huCO-scFv to bind to B cells derived from healthy donors (FIG. 6C), as well as showing that these fragments do not induce PCD in these same cell populations (FIG. 6D).

Induction of PCD by huCO-scFv

Induction of PCD was determined after 3 hours incubation with the huCO-scFv fragments, followed by staining with Annexin V and 7-AAD. Cells that stained positive for Annexin V were considered to be undergoing PCD, and as seen in FIG. 7, treatment of Jurkat cells with 0.1 or 1 μg/ml induced around 60% cell death after 3 hours.

Since the huCO-scFv fragments did not induce phagocytosis, possibly due to the lack of Fc part (data not shown), we engineered a monovalent scFv based on CO201, coupled with an Fc. This fragment will hereafter be termed CO201-scFv-Fc-mono. We evaluated the induction of hemagglutination and binding to RBCs of this fragment. CO201-scFv-Fc-mono did not induce hemagglutination at concentrations as high as 100 μg/ml (FIG. 8A), and had low binding to RBCs derived from three different donors (FIG. 8B).

Effect of huCO-scFv-Fc-Mono on Phagocytosis

### Materials and Methods

### Phagocytosis Assay

Cell lines were purchased from the American Type Culture Collection (ATCC). The Jurkat cell line (clone E6-1) was grown in RPMI1640, while RAW264.7 cells were grown in DMEM. Both cell culture medias were supplemented with 10% (v/v) FBS and 1% (v/v) PS.

### Staining of RAW264.7 Macrophages

RAW264.7 cells were rinsed once with supplemented DMEM, then stained with 40 nM Vybrant™ DiO Cell-Labelling Solution diluted in supplemented DMEM for 20 minutes in a standard cell incubator (humidified atmosphere with 95% air, 5% CO2 at 37° C.). After staining, the cells were rinsed three times with supplemented DMEM.

### Staining of Jurkat Cells and Antibody Treatment

Jurkat cells were collected by centrifugation, resuspended in DPBS, and stained with 1 μl/ml CellTrace™ Violet Cell Proliferation Dye for 20 minutes in a standard cell incubator. Non-incorporated CFSE was quenched by adding 5 times more supplemented RPMI and cells were pelleted and resuspended in supplemented DMEM. Target cells were added to the RAW264.7 tissue culture plates and treated with the indicated antibodies for 2 hours in a standard cell incubator, followed by two washes in PBS containing 1 mM EDTA. Cells were analyzed by flow cytometry and DiO⁺CellTrace⁺ cells represented phagocytosed target cells.

### Results

To determine the induction of phagocytosis by the huCO-scFv-Fc-mono, we incubated Jurkat cells in the presence of the murine macrophage cell line RAW264.7. Treatment with this fragment induced phagocytosis at equal levels as the full IgG4 molecule, CO-1.4 (FIG. 9).

### SPR Analysis of Antibodies Against Recombinant CD47 to Assess Binding Affinity

The binding affinity of the huCO-mAbs to CD47 was determined via surface plasmon resonance (SPR) on a Biacore S200 system. Recombinant biotinylated CD47 was immobilized on an SA streptavidin chip to a level of 100 RU (in 10 mM NaAc pH5.0). The huCO-mAbs reacted with recombinant CD47 at gradient concentrations.

### Analysis

### Instrument

### Biacore S200

### Materials and Reagents

SA chip (Streptavidine)·
recCD47: SinoBiological (12283-H27H-B); 17 kDa, C-term His- and AVI-tag, biotinylated
Antibodies: CO201-scFv (1.27 mg/ml), CO209-scFv (1.03 mg/ml), CO213-scFv (1.18 mg/ml). All antibodies stored at −80° C.
Buffer: 20 mM Phosphate buffer pH 7,4/2.7 mM KCl/137 mM NaCl/0,05% P20

### Single-Cycle Procedure

recCD47 immobilized to a level of 110-120 RU (in 10 mM NaAc pH 5.0)

Regeneration condition: 60 sec 10 mM glycin pH 2

Analyte (fragments) run in single-cycle mode

Injection order low to high concentration: 10 nM/5 nM/2.5 nM/1.25 nM/0.625 nM/0.3125 nM/0.1562 nM/0.07813 nM/0.039 nM

Injection order low to high

Flow 30 μl/min, 120 s on, 180 s off-1800s off after last injection

Temperature: 25° C.

### Results

A 1:1 binding model was used to fit the data. All curves are well fitted to the 1:1 model. Table 6 shows the values obtained for the binding on and off rates ka and kd, respectively. The affinity constant K_{D} is calculated as k_{d}/kₐ.

TABLE 6
Kinetic parameters for huCO-scFv

| Analyte | ka (1/Ms) | kd (1/s) | KD (M) | |
|---|---|---|---|---|
| CO201-scFv | 4.3 × 10⁶ | 2.6 × 10⁻⁴ | 6.1 × 10⁻¹¹ | 61 pM |
| CO209-scFv | 5.1 × 10⁶ | 1.7 × 10⁻⁴ | 3.3 × 10⁻¹¹ | 33 pM |
| CO213-scFv | 4.4 × 10⁶ | 2.1 × 10⁻⁴ | 4.8 × 10⁻¹¹ | 48 pM |

### Example 5: Activity of CO-201scFv In Vivo

### Materials and Methods

### CO201-scFv Antibody Fragment

The humanized single chain fragment variable (scFv) version of CO201, CO-201scFv, as described in Example 4 was produced by ATUM.

### Cell Cultures

All cell lines were purchased from American Type Culture Collection (ATCC). The B cell precursor acute lymphoblastic leukemia (BCP-ALL, hereafter ALL) cell line Reh was maintained at a density between 2×10⁵ and 1×10⁶ cells per ml in RPMI1640 medium (Lonza). HEK293T cells were used for lentiviral production and were subcultured every 2 to 3 days upon reaching 70 to 90% confluence in Dulbecco's Modified Eagle Medium (DMEM, ThermoFisher). Both cell culture medias were supplemented with 10% (v/v) fetal bovine serum (FBS, ThermoFisher) and 1% (v/v) penicillin/streptomycin (PS, ThermoFisher). The cells were cultured in a humidified atmosphere with 95% air and 5% CO2.

### Lentivirus Production in HEK293T Cells

Lentiviral vectors containing genes coding for firefly luciferase and enhanced green fluorescent protein (EGFP) were produced by transfecting HEK293T with 8.3 mg of each of the plasmids: pMD2.G envelope plasmid, pCMVΔ8.91 packaging plasmid, and pSLIEW transfer plasmid (1). Cells were cultured to 70% to 90% confluence at the day of transfection. The cell culture media was changed approximately 1 hour before transfection. Transfection mixtures were prepared using the Calcium Phosphate Transfection Kit (Invitrogen) according to manufacturer's instructions. Cell culture medium was changed 4 hours after transfection. Viral supernatants were collected after 2 days and concentrated using LentiX Concentrator (Takara Bio Inc.) at 4° C. overnight, before the viruses were collected by centrifugation at 1500 g for 45 minutes at 4° C. Pellets were suspended in a small volume (<1 mL) of cell culture medium and stored at −80° C. Frozen lentiviral stocks were titrated using Reh cells by using the standard transduction protocol (see the following section).

### Lentiviral Transduction of Reh Cells

Reh cells (5×10⁵ cells per well) were seeded into 48-well plates with 4 mg/mL polybrene (Merck Millipore) present in the cell culture media. Lentiviral concentrates were added to the cells, and spinfection was performed by centrifugating the plates at 900 g for 50 minutes at 34° C. After spinfection, plates were transferred to a standard cell incubator (37° C., 5% CO2 in a humidified atmosphere) for 2 days before removing the viral particles by 2 repeated washes at 300 g for 10 minutes at 4° C. A small aliquot was taken from these cells to analyze the amount of EGFP⁺ cells by flow cytometry. The remaining cells were subjected to intratibial (IT) injection into NSG mice.

### Establishment of ALL Xenograft Model

Transduced Reh cells (5×10⁵ cells) were injected into 6- to 8-week-old female NOD scid IL2Rγ^{null} (NSG) mice (The Jackson Laboratory) anesthetized with isoflurane (induction; 4% to 5%, maintenance; 2% to 3%, oxygen flow; 300 mL/min). The proximal end of the tibia was exposed as the knee was kept in a flexed position. A 23G needle was used to drill a hole into the tibia before injecting the Reh cells (40 μL per animal) using a 31G insulin syringe. Mice were treated with general and local analgesia; 0.05 mg/kg Temgesic (Schlering-Plough) and 1 to 2 mg/kg Marcain (AstraZeneca) before IT injection. General analgesia treatments were repeated 6 to 8 hours after IT injection. Mice were injected intraperitoneal (IP) with a single dose of 1.33 nM of either CO201-scFv or human IgG4 isotype control when the mice had reached a total flux above 10⁸ photons per seconds (p/s). Mice were housed under specific pathogenic-free conditions with food and water ad libitum. Health status was monitored daily, and all animal procedures were conducted according to the approval by the Norwegian Food Safety Authority under identification number 29016.

### In Vivo Imaging

Leukemic progression was monitored by noninvasive in vivo imaging using an IVIS Spectrum CT instrument (PerkinElmer). D-Luciferin (150 mg/kg, PerkinElmer) substrate was administered by intraperitoneal (IP) injection. After 9 minutes, 3 images were recorded with 1-minute intervals using the autoexposure setting. Mice were euthanized when showing heavy engraftment or symptoms above a predetermined humane end point.

### Results

Reh cells were stably transduced with the lentiviral firefly luciferase-EGFP vector and injected IT into NSG mice. Leukemic progression was followed by noninvasive in vivo imaging of the firefly luciferase expressing Reh cells until the mice displayed a satisfactory tumor take across all mice (>10⁸ p/s). The mice were divided into two groups and injected IP with 1.33 nM of either human IgG4 isotype control, or CO201-scFv immediately after the imaging procedure. Further IVIS were conducted at 3 and 6 days after treatment injection and revealed that mice treated with CO201-scFv had a significant reduction in tumor burden compared to mice treated with the isotype control (FIG. 10).

### REFERENCES

1. Bomken S, Buechler L, Rehe K, Ponthan F, Elder A, Blair H, et al. Lentiviral marking of patient-derived acute lymphoblastic leukaemic cells allows in vivo tracking of disease progression. Leukemia. 2013; 27 (3): 718-21.

### Example 6: SPR Analysis of CO201-scFv-Fc-Mono Against Recombinant CD47 to Assess Binding Affinity

The binding affinity of CO201-scFv-Fc-mono as described in Example 4 to CD47 was determined via surface plasmon resonance (SPR) on a Biacore S200 system. Recombinant biotinylated CD47 was immobilized on an SA streptavidin chip to a level of 100 RU (in 10 mM NaAc pH 5.0). CO201-scFv-Fc-mono reacted with recombinant CD47 at gradient concentrations

### Analysis

### Instrument: Biacore S200

### Material:

SA Chip (Streptavidine)
recCD47: SinoBiological (12283-H27H-B); 17 kDa, C-term His- and AVI-tag, biotinylated
Antibody Fragment: CO201-scFv-Fc-mono (77.3 kDa)
All proteins stored at −80° C.
Buffer: 20 mM Phosphate buffer pH 7.4/2.7 mM KCl/137 mM NaCl/0.05% P20

### Single-cycle Procedure:

recCD47 immobilized to a level of 100 RU (in 10 mM NaAc pH 5.0)
Regeneration condition: 60 sec 10 mM glycin pH 2
Analyte (CO201-scFv-Fc-mono) run in single-cycle mode
Injection order low to high concentration: 20 nM/10 nM/5 nM/2.5 nM/1.25 nM/0.625 nM/0.3125 nM/0.1562 nM/0.07813 nM/0.039 nM
Flow 30 μl/min, 120 s on-1800 s off after last injection
Temperature: 25° C.
Single-cycle mode

### Results

A 1:1 binding model was used to fit the data. All curves are well fitted to the 1:1 model. Table 7 shows the values obtained for the binding on and off rates Kₐ and K_{d}, respectively. The affinity constant K_{D} is calculated as K_{d}/Kₐ, 97 pM. Thus, the CO201-scFv-Fc-mono fusion protein shows a high binding affinity to CD47.

TABLE 7

| Analyte | kₐ (1/Ms) | k_{d} (1/s) | K_{D} (M) | |
|---|---|---|---|---|
| CO201- | 2.8 × 10⁷ | 2.7 × 10⁻³ | 9.7 × 10⁻¹¹ | 97 pM |
| scFv-Fc-mono | | | | |

1. A binding protein or antibody comprising one antigen binding domain that binds to CD47, said antigen binding domain comprising a heavy chain variable region that comprises three complementarity determining regions (CDRs), and a light chain variable region that comprises three CDRs, wherein said antigen binding domain binds to Q19, N45, T120, R121, E122 and G123 within CD47 as defined by SEQ ID NO:19, and wherein said binding protein or antibody binds monovalently to CD47.

2. The binding protein or antibody of claim 1, wherein said heavy chain variable region comprises:
(i) a variable heavy (VH) CDR1 that comprises the amino acid sequence of NFGMH (SEQ ID NO:5) or a sequence substantially homologous thereto,
(ii) a VH CDR2 that comprises the amino acid sequence of WINTYTGEPTYTDDFKG (SEQ ID NO:6) or a sequence substantially homologous thereto, and
(iii) a VH CDR3 that comprises the amino acid sequence of GDYRYGDS (SEQ ID NO:7) or a sequence substantially homologous thereto; and/or
wherein said light chain variable region comprises:
(iv) a variable light (V_{L}) CDR1 that comprises the amino acid sequence of RSSQSLVHSNGKTYLH (SEQ ID NO:8) or a sequence substantially homologous thereto,
(v) a V_{L} CDR2 that comprises the amino acid sequence of RVSNRFS (SEQ ID NO:9) or a sequence substantially homologous thereto, and
(vi) a V_{L} CDR3 that comprises the amino acid sequence of SQSTHVPFT (SEQ ID NO:10) or a sequence substantially homologous thereto;
wherein said substantially homologous sequence is a sequence containing 1, 2 or 3 amino acid substitutions compared to the given CDR sequence.

3. The binding protein or antibody of claim 1 or claim 2, wherein said heavy chain variable region comprises:
(i) a variable heavy (VH) CDR1 that comprises the amino acid sequence of NFGMH (SEQ ID NO:5),
(ii) a VH CDR2 that comprises the amino acid sequence of WINTYTGEPTYTDDFKG (SEQ ID NO:6), and
(iii) a VH CDR3 that comprises the amino acid sequence of GDYRYGDS (SEQ ID NO:7); and
wherein said light chain variable region comprises:
(iv) a variable light (V_{L}) CDR1 that comprises the amino acid sequence of RSSQSLVHSNGKTYLH (SEQ ID NO:8),
(v) a V_{L} CDR2 that comprises the amino acid sequence of RVSNRFS (SEQ ID NO:9), and
(vi) a V_{L} CDR3 that comprises the amino acid sequence of SQSTHVPFT (SEQ ID NO:10).

4. The binding protein or antibody of any one of claims 1 to 3, wherein the heavy chain variable region comprises the amino acid sequence of SEQ ID NO:3, or a sequence having at least 80% sequence identity thereto, and/or wherein the light chain variable region comprises the amino acid sequence of SEQ ID NO:4, or a sequence having at least 80% sequence identity thereto; or wherein the binding protein or antibody comprises a humanized version of SED ID NO: 3 and/or SEQ ID NO:4; or wherein the heavy chain variable region comprises the amino acid sequence of one or more of SEQ ID NO:22, 23, 24, 25, or 26, or a sequence having at least 80% sequence identity thereto, and/or wherein the light chain variable region comprises the amino acid sequence of SEQ ID NO:27, 28 or 29, or a sequence having at least 80% sequence identity thereto.

5. The antibody of any one of claims 1 to 4, wherein said antibody is an scFv antibody, or a monovalent scFv-Fc format.

6. The binding protein or antibody of any one of claims 1 to 5, which has a binding affinity (K_{D}) for CD47 of less than 250 pM, preferably less than 200 pM.

7. The binding protein or antibody of any one of claims 1 to 6, wherein said binding protein or antibody is capable of inducing programmed cell death of tumour cells and/or does not induce haemagglutination of red blood cells.

8. An immunoconjugate comprising the binding protein or antibody of any one of claims 1 to 7, operatively attached to at least one other therapeutic or diagnostic agent.

9. One or more nucleic acid molecules comprising nucleotide sequences that encode the binding protein or antibody or immunoconjugate of any one of claims 1 to 8.

10. One or more expression vectors comprising the one or more of the nucleic acid molecules of claim 9.

11. One or more host cells or viruses comprising said expression vectors of claim 10, or said nucleic acid molecules of claim 9, or expressing the antibody or binding protein or immunoconjugate of any one of claims 1 to 8.

12. A method of producing the binding protein or antibody or immunoconjugate of any one of claims 1 to 8, said method comprising the steps of (i) culturing a host cell comprising the expression vectors of claim 10 or the nucleic acid molecules of claim 9, under conditions suitable for the expression of the encoded binding protein or antibody or immunoconjugate; and optionally (ii) isolating or obtaining the binding protein or antibody or immunoconjugate from the host cell or from the growth medium/supernatant.

13. A composition comprising the binding protein or antibody of any one of claims 1 to 7, the immunoconjugate of claim 8, the one or more nucleic acid molecules of claim 9, the one or more expression vectors of claim 10, or the one or more host cells or viruses of claim 11.

14. The binding protein or antibody of any one of claims 1 to 7, the immunoconjugate of claim 8, the one or more nucleic acid molecules of claim 9, the one or more expression vectors of claim 10, the one or more host cells or viruses of claim 11, or the composition of claim 13, for use in therapy, preferably for use in the treatment or prevention of cancer.

15. A binding protein or antibody comprising one antigen binding domain that binds to CD47, wherein said antigen binding domain is in an scFv format, and wherein said antigen binding domain is fused or connected or otherwise attached to an Fc region, and wherein said binding protein or antibody binds monovalently to CD47.

16. The binding protein or antibody of claim 15, wherein said binding protein or antibody is as defined in any one of claims 1 to 7.

17. The binding protein or antibody of claim 15 or claim 16, for use in therapy, preferably for use in the treatment or prevention of cancer.

18. A method of treating or preventing cancer, which method comprises administering to a patient in need thereof a therapeutically effective amount of the binding protein or antibody of any one of claims 1 to 7 or claim 15, the immunoconjugate of claim 8, the one or more nucleic acid molecules of claim 9, the one or more expression vectors of claim 10, the one or more host cells or viruses of claim 11, or the composition of claim 13.

19. The use of a binding protein or antibody of any one of claims 1 to 7 or claim 15, the immunoconjugate of claim 8, the one or more nucleic acid molecules of claim 9, the one or more expression vectors of claim 10, the one or more host cells or viruses of claim 11, or the composition of claim 13, in the manufacture of a medicament for use in therapy, preferably for use in the treatment or prevention of cancer.


SEQUENCE LISTING (36 sequences)

SEQ ID NO: 1 - DNA, 351 bp, synthetic construct
  source 1..351: mol_type = other DNA
cagatccagttggtgcagtctggacctgaactgaagaagcctggagagacagtcaagatctcctgcaaggcttctgggtataccttcacaaactttggaatgcactgggtgaagcaggctccagggaagggtttaaagtggatgggctggataaacacctacactggagagccaacatatactgatgacttcaagggacggtttgccttctctttggagacctctgccagcactgcctatttgcagatcaacagcctcaaaaatgaggacacggctacgtttttctgtacaagaggggattataggtacggtgactcctggggccaaggcaccactctcacagtctcctca

SEQ ID NO: 2 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacacagtaatggaaaaacctacttacattggtacctgcagaagccaggccagtctccaaagctcctgatctacagagtttccaaccgattttctggggtcccagacaggttcagtggcagtggatcagggacagatttcacactcaagatcagcagaatagaggctgaggatctgggagtttatttctgctctcaaagtacacatgttccattcacgttcggctcggggacaaagttggaaataaaa

SEQ ID NO: 3 - protein, 117 aa, synthetic construct
QIQLVQSGPELKKPGETVKISCKASGYTFTNFGMHWVKQAPGKGLKWMGWINTYTGEPTYTDDFKGRFAFSLETSASTAYLQINSLKNEDTATFFCTRGDYRYGDSWGQGTTLTVSS

SEQ ID NO: 4 - protein, 112 aa, synthetic construct
DVVMTQTPLSLPVSLGDQASISCRSSQSLVHSNGKTYLHWYLQKPGQSPKLLIYRVSNRFSGVPDRFSGSGSGTDFTLKISRIEAEDLGVYFCSQSTHVPFTFGSGTKLEIK

SEQ ID NO: 5 - protein, 5 aa, synthetic construct
NFGMH

SEQ ID NO: 6 - protein, 17 aa, synthetic construct
WINTYTGEPTYTDDFKG

SEQ ID NO: 7 - protein, 8 aa, synthetic construct
GDYRYGDS

SEQ ID NO: 8 - protein, 16 aa, synthetic construct
RSSQSLVHSNGKTYLH

SEQ ID NO: 9 - protein, 7 aa, synthetic construct
RVSNRFS

SEQ ID NO: 10 - protein, 9 aa, synthetic construct
SQSTHVPFT

SEQ ID NO: 11 - protein, 30 aa, synthetic construct
QIQLVQSGPELKKPGETVKISCKASGYTFT

SEQ ID NO: 12 - protein, 14 aa, synthetic construct
WVKQAPGKGLKWMG

SEQ ID NO: 13 - protein, 32 aa, synthetic construct
RFAFSLETSASTAYLQINSLKNEDTATFFCTR

SEQ ID NO: 14 - protein, 11 aa, synthetic construct
WGQGTTLTVSS

SEQ ID NO: 15 - protein, 23 aa, synthetic construct
DVVMTQTPLSLPVSLGDQASISC

SEQ ID NO: 16 - protein, 15 aa, synthetic construct
WYLQKPGQSPKLLIY

SEQ ID NO: 17 - protein, 32 aa, synthetic construct
GVPDRFSGSGSGTDFTLKISRIEAEDLGVYFC

SEQ ID NO: 18 - protein, 10 aa, synthetic construct
FGSGTKLEIK

SEQ ID NO: 19 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 20 - protein, 15 aa, synthetic construct
GGGGSGGGGSGGGGS

SEQ ID NO: 21 - protein, 244 aa, synthetic construct
QIQLVQSGPELKKPGETVKISCKASGYTFTNFGMHWVKQAPGKGLKWMGWINTYTGEPTYTDDFKGRFAFSLETSASTAYLQINSLKNEDTATFFCTRGDYRYGDSWGQGTTLTVSSGGGGSGGGGSGGGGSDVVMTQTPLSLPVSLGDQASISCRSSQSLVHSNGKTYLHWYLQKPGQSPKLLIYRVSNRFSGVPDRFSGSGSGTDFTLKISRIEAEDLGVYFCSQSTHVPFTFGSGTKLEIK

SEQ ID NO: 22 - protein, 117 aa, synthetic construct
QVQLVQSGSELKKPGASVKVSCKASGYTFTNFGMHWVRQAPGQGLEWMGWINTYTGEPTYTDDFKGRFVFSLDTSVSTAYLQISSLKAEDTAVYYCARGDYRYGDSWGQGTTVTVSS

SEQ ID NO: 23 - protein, 117 aa, synthetic construct
QVQLVQSGSELKKPGASVKVSCKASGYTFTNFGMHWVRQAPGQGLEWMGWINTYTGEPTYTDDFKGRFVFSLDTSVSTAYLQISSLKAEDTAVYYCTRGDYRYGDSWGQGTTVTVSS

SEQ ID NO: 24 - protein, 117 aa, synthetic construct
QVQLVQSGSELKKPGASVKVSCKASGYTFTNFGMHWVRQAPGQGLKWMGWINTYTGEPTYTDDFKGRFVFSLDTSVSTAYLQISSLKAEDTAVYYCTRGDYRYGDSWGQGTTVTVSS

SEQ ID NO: 25 - protein, 117 aa, synthetic construct
QVQLVQSGSELKKPGASVKVSCKASGYTFTNFGMHWVRQAPGKGLKWMGWINTYTGEPTYTDDFKGRFVFSLDTSVSTAYLQISSLKAEDTAVYYCTRGDYRYGDSWGQGTTVTVSS

SEQ ID NO: 26 - protein, 117 aa, synthetic construct
QVQLVQSGSELKKPGASVKVSCKASGYTFTNFGMHWVRQAPGQGLKWMGWINTYTGEPTYTDDFKGRFVFSLDTSVSTAYLQISSLKAEDTAVYYCARGDYRYGDSWGQGTTVTVSS

SEQ ID NO: 27 - protein, 112 aa, synthetic construct
DIVMTQTPLSLSVTPGQPASISCRSSQSLVHSNGKTYLHWYLQKPGQPPQLLIYRVSNRFSGVPDRFSGSGSGTDFTLKISRVEAEDVGVYYCSQSTHVPFTFGQGTKLEIK

SEQ ID NO: 28 - protein, 112 aa, synthetic construct
DIVMTQTPLSLSVTPGQPASISCRSSQSLVHSNGKTYLHWYLQKPGQSPQLLIYRVSNRFSGVPDRFSGSGSGTDFTLKISRVEAEDVGVYYCSQSTHVPFTFGQGTKLEIK

SEQ ID NO: 29 - protein, 112 aa, synthetic construct
DIVMTQTPLSLSVTPGQPASISCRSSQSLVHSNGKTYLHWYLQKPGQSPKLLIYRVSNRFSGVPDRFSGSGSGTDFTLKISRVEAEDVGVYYCSQSTHVPFTFGQGTKLEIK

SEQ ID NO: 30 - protein, 218 aa, synthetic construct
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 228 aa, synthetic construct
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 228 aa, synthetic construct
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 33 - protein, 10 aa, synthetic construct
SKYGPPCPSC

SEQ ID NO: 34 - protein, 472 aa, synthetic construct
QVQLVQSGSELKKPGASVKVSCKASGYTFTNFGMHWVRQAPGQGLEWMGWINTYTGEPTYTDDFKGRFVFSLDTSVSTAYLQISSLKAEDTAVYYCARGDYRYGDSWGQGTTVTVSSGGGGSGGGGSGGGGSDIVMTQTPLSLSVTPGQPASISCRSSQSLVHSNGKTYLHWYLQKPGQPPQLLIYRVSNRFSGVPDRFSGSGSGTDFTLKISRVEAEDVGVYYCSQSTHVPFTFGQGTKLEIKSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 228 aa, synthetic construct; identical to SEQ ID NO: 32

SEQ ID NO: 36 - protein, 10 aa, synthetic construct
SKYGPPCPPC